(19) United States
(12) Patent Application Publication
(10) Pub. No.: US 2025/0145767 A1
(43) Pub. Date: May 8, 2025

(54) HEXAVALENT SUBSTITUTED BENZENES

(71) Applicant: Northeastern University, Boston, MA (US)

(72) Inventors: Steven Lustig, Attleboro, MA (US); Kätchen Kelly Lachmayr, Landenberg, PA (US)

(21) Appl. No.: 18/838,167

(22) PCT Filed: Feb. 16, 2023
(86) PCT No.: PCT/US23/13212

§ 371 Date: Aug. 13, 2024

Related U.S. Application Data
(60) Provisional application No. 63/399,827, filed on Aug. 22, 2022.
(60) Provisional application No. 63/310,780, filed on Feb. 16, 2022.

(51) Int. Cl. C08G 69/32; C07C 225/22; C08G 73/02; D01F 6/80

(52) U.S. Cl. CPC C08G 69/32; C07C 225/22; C08G 73/02; D01F 6/805

(57) ABSTRACT

Provided herein are hexavalent, fully-functionalized benzene derivatives comprising two types of functional groups, wherein each functional group type alternates around the benzene ring, so no identical functional groups are positioned next to one another. In certain embodiments, the first functional group is selected from an aldehyde (CO), imine (CNH), or iminium, and the second functional group is selected from a primary amine (NH₂) or ammonium (NH₃⁺). The hexavalent, fully-functionalized benzene derivatives of the invention as useful as monomers in the formation of planar, two-dimensional polymer networks. Also provided are covalent organic frameworks comprising the hexavalent, fully-functionalized benzene derivatives of the invention. Also provided herein are synthetic fibers comprising such two-dimensional polymers. Further provided are methods of making the hexavalent, fully-functionalized benzene derivatives.

### RELATED APPLICATIONS

This application claims the benefit of priority to U.S. Provisional Patent Application No. 63/310,780, filed Feb. 16, 2022; and U.S. Provisional Patent Application No. 63/399,827, filed Aug. 22, 2022.

### GOVERNMENT SUPPORT

This invention was made with government support under Grant Number W911NF-20-2-0024 awarded by the U.S. Army Research Office. The government has certain rights in the invention.

### BACKGROUND

Materials like Kevlar® represent the current standard for light weight, high-performance materials. Kevlar® falls into the class of poly(p-phenylene terephthalamide) (PPTA) materials, where these polymer fibers are among the stiffest and strongest engineered materials per unit mass that are currently available.¹ These PPTA fibers possess a unique combination of high stiffness and strength with resistance to elevated temperature, chemicals, ultraviolet, moisture, and creep; enabled by two key features of the polymer; (1) the rigid aromatic backbone that provides stiffness and strength to the molecule, and (2) strong intermolecular hydrogen bonding that provides macroscopic strength and stiffness.¹

Inspired from the strong aromatic networks in PPTA materials, a new class of high-performance materials has been re-envisioned, now as extend planer sheets or two-dimensional (2D)polymers. Similar to covalent organic frameworks (COFs), which are layered, porous, periodic structures (crystalline) formed from polymer networks.² Traditional 2D COFs have been designed with a relatively sparse covalent network, for tailored porosity that allows for selective chemical separations, but also results diminished mechanical properties like lower molecular strength and stiffness compared to denser molecular networks, like graphene or 2D polymers.³ Monomers with six-fold functionalities, allows for the creation of extended networks, compared to bi-directional fibers, producing higher density networks' A new material, ‘graphamid’, is composed of six amide bonds, with strong aromatic, high-density networks, and contains strong hydrogen bonding networks between molecules. These features are thought to generate high strength and stiffness at low molecular weights.³ There is a need to develop analogs of graphamid. Doing so requires hexavalent benzene ring monomers.

Production of hexavalent or fully functionalized benzene rings produces numerous synthetic challenges, as each position on a central six-carbon membered aromatic ring structure contains a functionalized group, or substituents consisting of atoms other than carbon and/or hydrogen. Although a single set of functional groups on alternating positions may be targeted synthetically, the addition of alternating reactive functional groups occupying every position of a six-member ring presents unique challenges. Past attempts have utilized metal-catalysts for coupling reactions, e.g., using free or protected amines, where protective groups have been utilized to limit side-reaction or decommission during synthesis since amines are particularly reactive groups. The addition of amines with aldehydes is particularly challenging because of the assumed reactivity between the two functional groups. Additionally, fully functionalized benzene rings present additional challenges because of steric and torsional strain within a small, confined space that makes multiple additions to a central ring, and achieving full functionalization, particularly difficult.

Thus, the specifications of the monomers that form graphamid analogs present numerous synthetic challenges, in controlling the positional selectivity, so that each functionality alternates the other, but also the selection and utilization of protective groups to prevent neighboring groups from spontaneously reacting, all while minimizing contribution to steric-strain round around the central benzene ring. Furthermore, any protective groups used must be easily removable, as to not impede any subsequent transamination for the polymerization into graphamid analogs. There is a need to develop hexavalent or fully functionalized benzene rings that meet these criteria.

### SUMMARY OF THE INVENTION

In certain aspects, the invention provides a compound of formula (I):

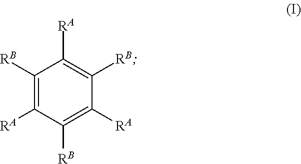

or a salt thereof;
wherein:
R^{A} is selected from the group consisting of —CH═O, —CH═NH, and —CH═NH₂⁺;
R^{B} is selected from the group consisting of —NH₂, —NH₃⁺, and —N═C(R^{C})₂; and
each occurrence of R^{C} is independently selected from the group consisting of optionally substituted aryl and heteroaryl;
all occurrences of R^{A} are the same; and
all occurrences of R^{B} are the same.

In certain aspects, the invention provides a two-dimensional polymer formed from a plurality of hexavalent monomer units; wherein the hexavalent monomer unit is a compound of formula (I), wherein R^{B} is —NH₂ or —NH₃⁺; or

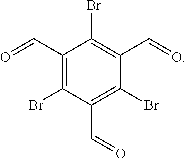

In certain embodiments, the invention provides a one-dimensional polymer formed from a plurality of hexavalent monomer units; wherein the hexavalent monomer unit is a compound of formula (I), wherein R^{B} is —NH₂ or —NH₃⁺; or

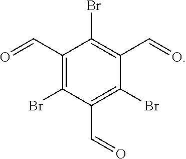

In certain aspects, the invention provides a synthetic fiber comprising a two-dimensional polymer of the invention.

The present invention also provides a copolymer comprising the two-dimensional polymer of the invention and the one-dimensional polymer of the invention.

In other aspects, the invention provides a compound of formula (I-PG):

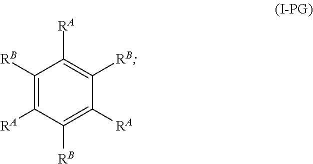

or a salt thereof;
wherein:
R^{A} is selected from the group consisting of —CH═O, —CH═NH, —CH═NH₂⁺, a protected aldehyde, and a protected imine;
R^{B} is selected from the group consisting of —NH₂, —NH₃⁺, —N═C(R^{C})₂, and a protected amine; and
each occurrence of R^{C} is independently selected from the group consisting of optionally substituted aryl and heteroaryl;
wherein:
   (a) at least one instance of R^{A} is a protected aldehyde or a protected imine; or
   (b) at least one instance of R^{B} is a protected amine.

The invention also provides a method of making compound (2):

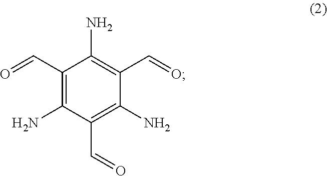 [SMILES: Nc1c(C=O)c(N)c(C=O)c(N)c1C=O]

comprising the step of combining under conditions sufficient to produce compound (2): compound (2i) and a Bronsted acid; wherein compound (2i) has the following structure:

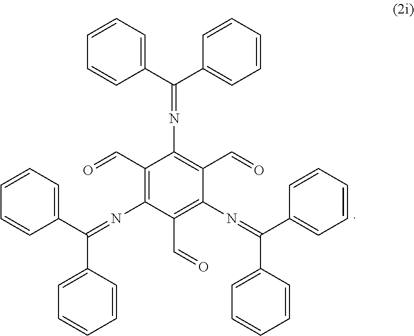 [SMILES: O=Cc1c(N=C(c2ccccc2)c2ccccc2)c(C=O)c(N=C(c2ccccc2)c2ccccc2)c(C=O)c1N=C(c1ccccc1)c1ccccc1]

In certain embodiments, the invention provides a method of making any one of compounds (2)-(7), or a salt thereof, comprising the step of combining under conditions sufficient to produce any one of compounds (2)-(7):
compound (1) and a nucleophilic amine source; wherein compound (1) has the following structure:

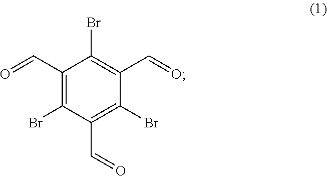 [SMILES: O=Cc1c(Br)c(C=O)c(Br)c(C=O)c1Br]

and
   the amine source is selected from the group consisting of ammonia (NH₃), and salts thereof, ammonium salts, primary organic amines, and salts thereof.

The invention also provides a method of making a two-dimensional polymer of the invention, comprising the step of combining compound of formula (I), wherein R^{B} is —NH₂ or —NH₃⁺; or

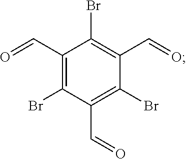 [SMILES: O=Cc1c(Br)c(C=O)c(Br)c(C=O)c1Br]

and
H₂N-L-NH₂, wherein L represents the linking moiety;
   wherein the conditions sufficient to produce the two-dimensional polymer comprise a Bronsted acid.

### BRIEF DESCRIPTION OF THE DRAWINGS

FIG. 1 depicts the covalent organic frameworks that are accessible through the hexa-substituted benzene compounds of the invention.

FIG. 2 shows additional analyses of compound 2i, specifically differential scanning calorimetry (DSC), thermogravimetric analysis (TGA), MALDI-TOF, and GC-MS Pyrrolysis.

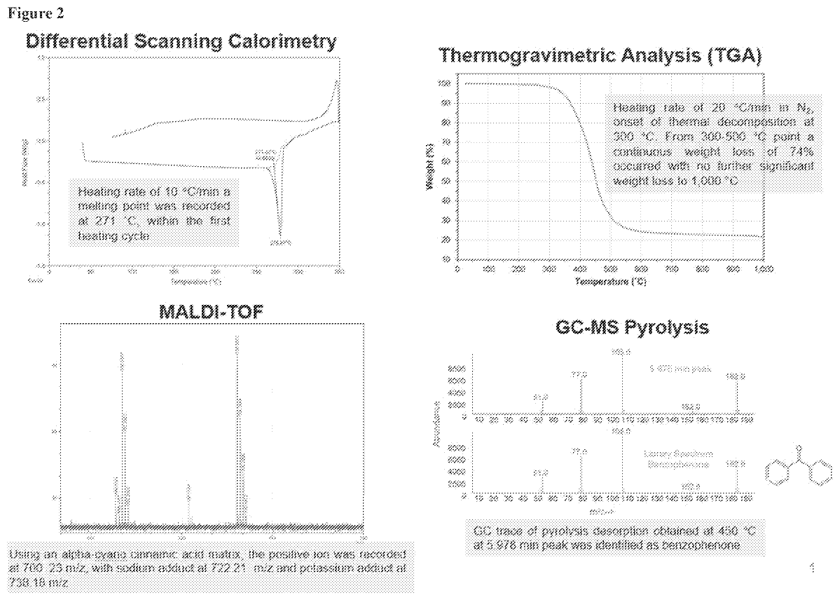

FIG. 3 shows the results of FTIR analysis of Graphimine produced by acid catalysis with 20 equivalents of acetic acid.

FIG. 4 shows the results of FTIR analysis of Graphimine produced by acid catalysis with 10 equivalents of para-toluenesulfonic acid.

FIG. 5 shows the results of FTIR analysis of 1,4-diphenylimine linked COF produced by acid catalysis from compound 2.

FIG. 6 shows the results of FTIR analysis of the reaction mixture subjecting graphimine to oxidation conditions.

### DETAILED DESCRIPTION OF THE INVENTION

This invention is based on the discovery of a new class of fully-functionalized hexavalent benzene derivatives that are useful as monomer building blocks to access planar, two-dimensional polymers. An exemplary planar, two-dimensional polymer that is described herein is ‘graphimine’, which is expected to have superior electrical conductivity relative to graphamid.⁴ Graphimine, as a 2D polymer and a new class of high performance materials is expected to offer superior strength to weight ratios, superior stiffness to weight ratios, superior electrical conductivity, and high thermal stability as compared to current industry standards. However, the synthesis of materials such as graphimine is not trivial. In order to access graphimine and related materials, the inventors designed novel monomer species with two different reactive functionalities on alternating positions around a central benzene ring.

Hexavalent. Fully-Functionalized Benzene Derivatives

The present invention provides the successful synthesis and isolation of 2,4,6-triaminobenzene-1,3,5-tricarbaldehyde (2), a novel compound, which contains aldehyde functionalities in the 1, 3, and 5 positions, and amines functionalities in the 2, 4, and 6 positions. The present invention further provides the prophetic production of five additional compounds: 2,4,6-tris(iminomethyl)benzene-1,3,5-triamine (3), 2,4,6-triformylbenzene-1,3,5-triaminium (4), 2,4,6-tris(iminomethyl)benzene-1,3,5-triaminium (5), 2,4,6-tris(phenylmethaniminium)benzene-1,3,5-triamine (6), and 2,4,6-tris(phenylmethaniminium)benzene-1,3,5-triaminium (7), that may contain organic or inorganic counter ions. See Scheme 1 for Compounds 1-7.

The hexavalent benzene derivatives of the invention comprise two-types of functional groups, where each functional group type alternates around the benzene ring, so no identical functional groups are positioned next to one another, and the functional groups may include aldehydes (CO), imines (CNH), or iminiums as the first functional group type, and either primary amines (NH₂) or ammoniums (NH₃⁺) as the second functional group type.

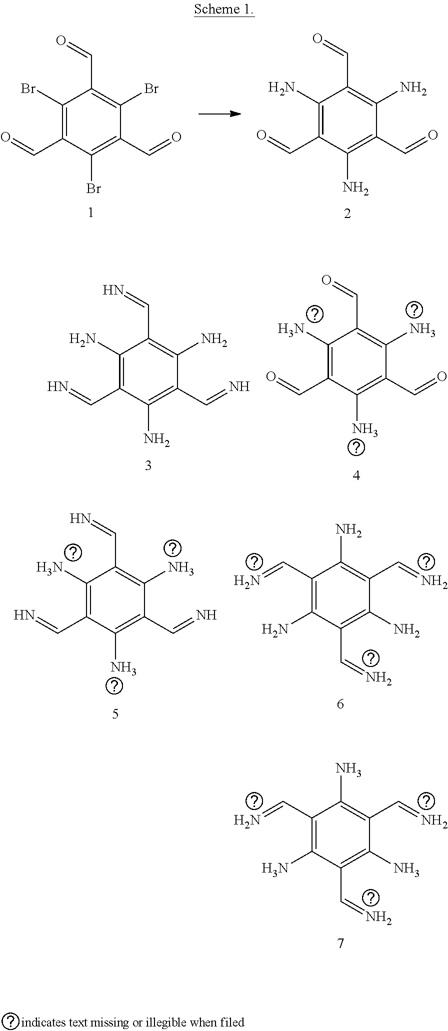

### Depiction of Compounds: 2,4,6-tribromobenzene-1,3,5-tricarbaldehyde (1), 2,4,6-triaminobenzene-1,3,5-tricarbaldehyde (2), 2,4,6-tris(iminomethyl)benzene-1,3,5-triamine (3), 2,4,6-triformylbenzene-1,3,5-triaminium (4), 2,4,6-tris(iminomethyl)benzene-1,3,5-triaminium (5), 2,4,6-tris(phenylmethaniminium)benzene-1,3,5-triamine (6), and 2,4,6-tris(phenylmethaniminium)benzene-1,3,5-triaminium (7), without Counter Ions

In certain embodiments, the invention provides a compound of formula (I):

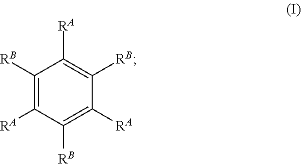

or a salt thereof;
wherein:
R^{A} is selected from the group consisting of —CH═O, —CH═NH, and —CH═NH₂⁺;
R^{B} is selected from the group consisting of —NH₂, —NH₃⁺, and —N═C(R^{C})₂; and
each occurrence of R^{C} is independently selected from the group consisting of optionally substituted aryl and heteroaryl;
all occurrences of R^{A} are the same; and
all occurrences of R^{B} are the same.

In certain embodiments, R^{A} is —CH═O. In alternative embodiments, R^{A} is —CH═NH.

In yet further alternative embodiments, R^{A} is —CH═NH₂⁺.

In certain embodiments, R^{B} is —NH₂ or —NH₃⁺. In certain embodiments, R^{B} is —NH₂. In alternative embodiments, R^{B} is —NH₃⁺.

In certain embodiments, the compound of the invention is selected from the following compounds:

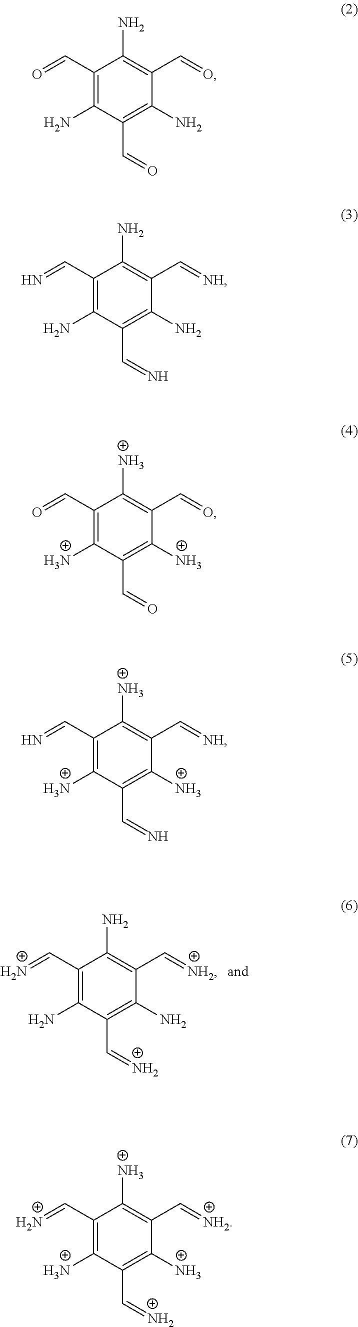

In certain preferred embodiments, the compound is compound (2).

In other embodiments, R^{B} is —N═C(R^{C})₂. In certain such embodiments, each occurrence of R^{C} is independently optionally substituted aryl, preferably each occurrence of R^{C} is phenyl.

In certain embodiments, the compound is

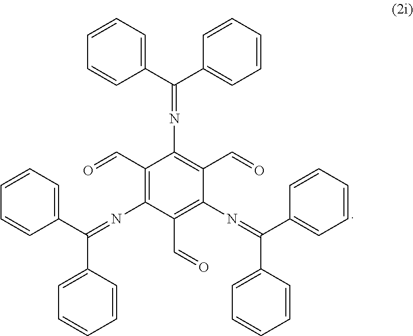 [SMILES: O=Cc1c(N=C(c2ccccc2)c2ccccc2)c(C=O)c(N=C(c2ccccc2)c2ccccc2)c(C=O)c1N=C(c1ccccc1)c1ccccc1]

In certain embodiments, a person skilled in the art may wish to mask one or more of the reactive groups in a hexavalent benzene derivative so that the compound can undergo a chemoselective transformation. Common strategies used to mask a reactive group include the use of a protecting group on a reactive moiety.

In certain embodiments, the invention provides a compound of formula (I-PG):

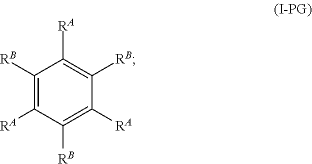

or a salt thereof;
wherein:
R^{A} is selected from the group consisting of —CH═O, —CH═NH, —CH═NH₂⁺, a protected aldehyde, and a protected imine;
R^{B} is selected from the group consisting of —NH₂, —NH₃⁺, —N═C(R^{C})₂, and a protected amine; and
each occurrence of R^{C} is independently selected from the group consisting of optionally substituted aryl and heteroaryl;
wherein:
   (a) at least one instance of R^{A} is a protected aldehyde or a protected imine; or
   (b) at least one instance of R^{B} is a protected amine.

In certain embodiments, a protected aldehyde means an aldehyde protected by a protecting group. Exemplary protecting groups for aldehydes include acetals. In certain embodiments, R^{A} is a protected aldehyde, and the protected aldehyde is an acetal having the structure —CH(O(hydrocarbyl))₂. In certain embodiments, the protected aldehyde is —CH(O(CH₂)ₙ)O), wherein n is 2 or 3.

In certain embodiments, a protected imine means an imine wherein the nitrogen is protected by a non-hydrogen substituent. In certain embodiments, R^{A} is a protected imine, and the protected imine has the structure —CH═N(alkyl).

In certain embodiments, a protected amine means an amine protected by a protecting group. Exemplary protecting groups for amines include carbobenzyloxy, tert-butyloxycarbonyl, acetyl, benzoyl, benzyl, carbamate, and tosyl. In some embodiments, R^{B} is a protected amine selected from the group consisting of —NH(C═O)O(hydrocarbyl), —NH(arylalkyl), and —NH(C═O)hydrocarbyl.

### Two-Dimensional Polymers

The hexavalent fully-functionalized benzene derivatives described above, such as Compounds 2-7, are as useful as monomer units that allow for polymerization into two-dimensional polymer networks by taking advantage of the full functionalization that allows for polymerization to occur in all directions. This enables access to platelet polymers, rather than the commonly observed linear chain polymers, bottle brush polymers, or Miktoarm polymers. Two-dimensional polymers are expected to have unparalleled mechanical strength, stiffness, and toughness, and resistance to thermal exposure and creep. The class of two-dimensional polymers that utilizes compounds 2-7 has drawn inspiration from materials like graphene and Kevlar, taking advantage of an extended aromatic backbone that is responsible for molecular strength, while allowing for some of the lowest mass-to-density ratios. One key advantage of using hexavalent monomers is the small pore size that will result from the polymerization of these species. As compared to existing covalent organic frameworks (COFs), these monomers will have dramatically decreased pore size. Such polymers do not require the use of a linking component to connect between monomer units, though, in certain embodiments, the use of a linking moiety may be desired.

Additionally, the selective use of aldehyde, amine, imine, iminium, and ammonium functionalities allows for reversible transamination between the Schiff base linkages, allowing for intermolecular rearrangement and optimization of the polymer spatial orientation during synthesis. This enables the formation of compact pores without necessarily requiring the use of linkers between monomer units. Imine-linked COFs are typically prepared through the condensation of aryl-amines and aldehydes under acidic conditions which promotes dynamic imine exchange. COFs linked with imines are generally more chemically stable than their boron-linked counterparts, making them more promising for a broad range of applications, such as energy storage devices, proton-conductive membranes, selective filiations devices, and catalyst supports.

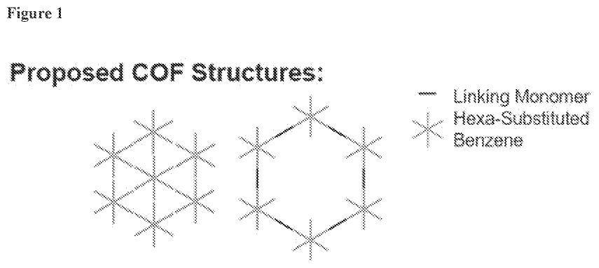

The hexa-substituted benzene derivatives described herein enable access to two-dimensional covalent organic frameworks (COFs), by taking advantage of the molecules' high degree of symmetry, functionality, and planarity. Covalent organic frameworks (COFs) are crystalline, porous networks made from non-metal elements that offer well-defined solid-state structures and tunable pore sizes, which emerge from the topology and shape of their molecular building blocks or monomer units. In certain embodiments, the COFs resulting from the compounds of the invention comprise triangular pores. In alternative embodiments, the COFs resulting from the compounds of the invention comprise hexagonal pores. The COFs may comprise additional pendent functionalities. The proposed COF structures are depicted in FIG. 1.

In certain embodiments, the invention provides a two-dimensional polymer formed from a plurality of hexavalent monomer units; wherein the hexavalent monomer unit is a compound of formula (I), wherein R^{B} is —NH₂ or —NH₃⁺; or

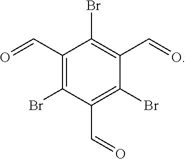

In certain embodiments, the hexavalent monomer units are covalently linked. In certain such embodiments, the hexavalent monomer units are covalently linked directly to each other, without a linking moiety to connect the monomer units.

In other embodiments, the hexavalent monomer units are covalently linked through a linking moiety. Linking moieties that are divalent (i.e., that connect two monomer units) are often referred to in the art as linkers. Linking moieties that have valency higher than two (e.g., trivalent or tetravalent linking moieties) are referred to in the art as nodes.

In some embodiments, each linking moiety has a valency of two or greater. For example, in some embodiments, each linking moiety is independently divalent, trivalent, or tetravalent.

In some embodiments, each linking moiety independently comprises one or more optionally substituted aromatic rings.

In certain embodiments, each linking moiety is independently selected from the group consisting of

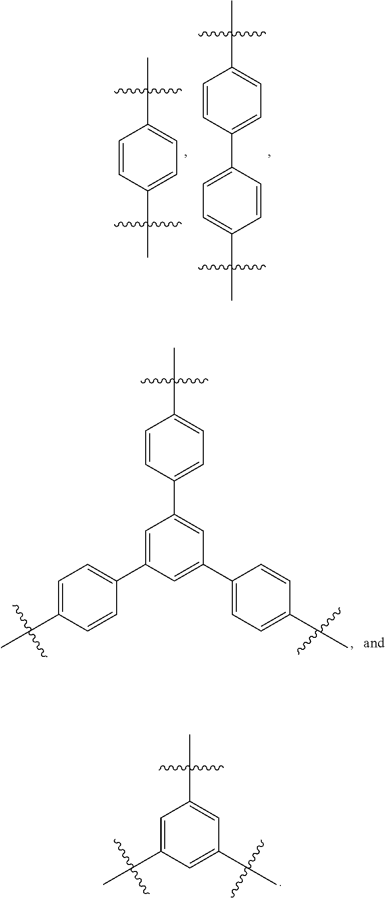

In certain such embodiments, all linking moieties are the same.

In certain embodiments, each linking moiety is

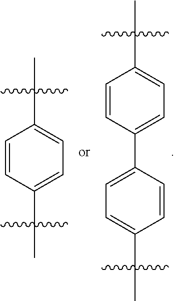

In certain embodiments, the two-dimensional polymer has a structure selected from:

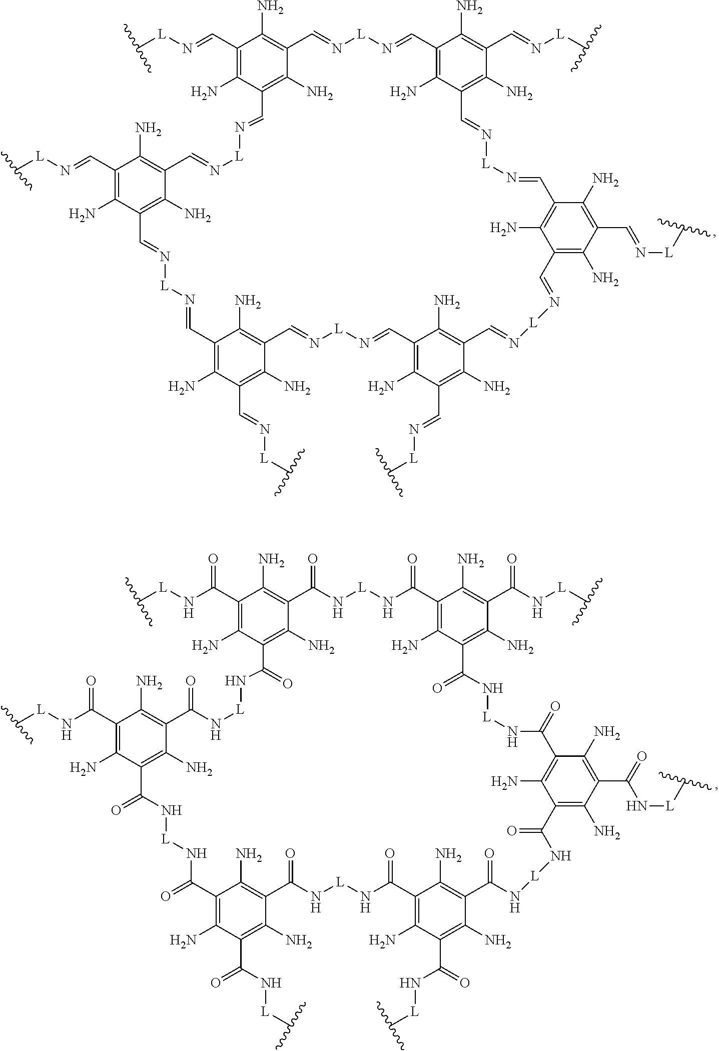

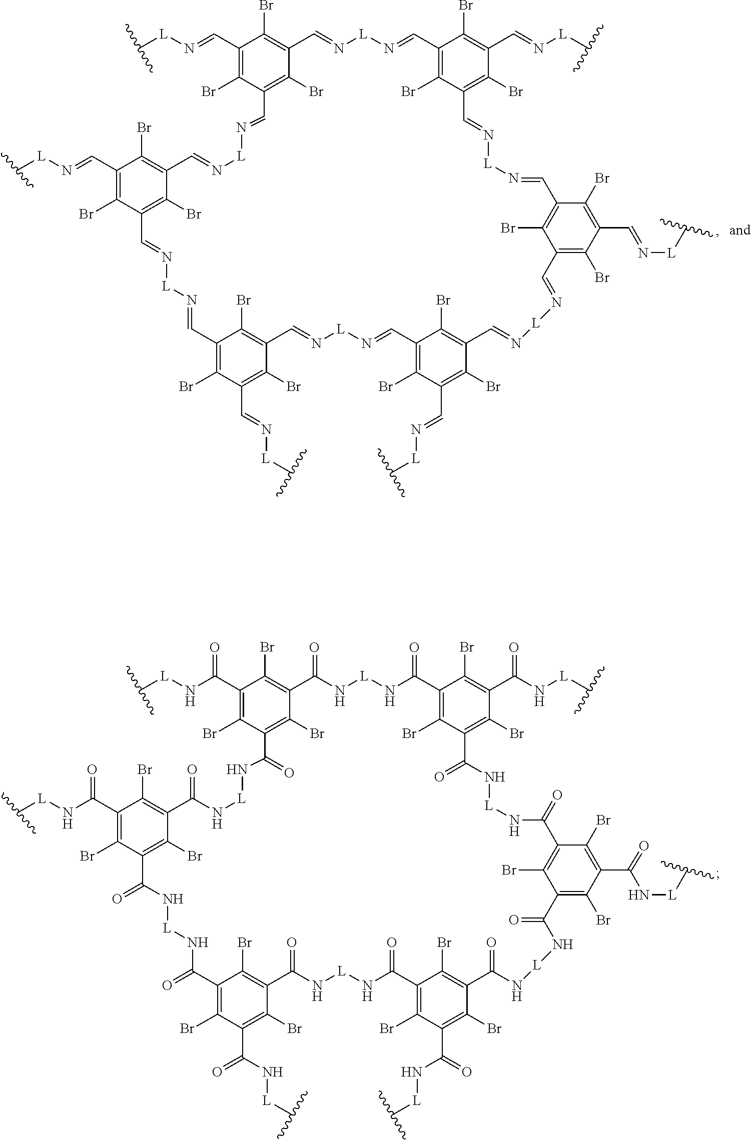

wherein each occurrence of L is

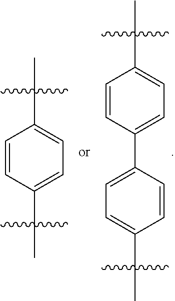

In other embodiments, each linking moiety is

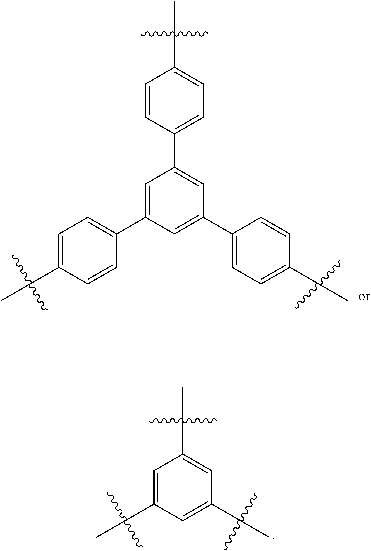 [SMILES: *c1ccc(-c2cc(-c3ccc(*)cc3)cc(-c3ccc(*)cc3)c2)cc1 ; *c1cc(*)cc(*)c1]

In certain embodiments, the two-dimensional polymer has a structure selected from

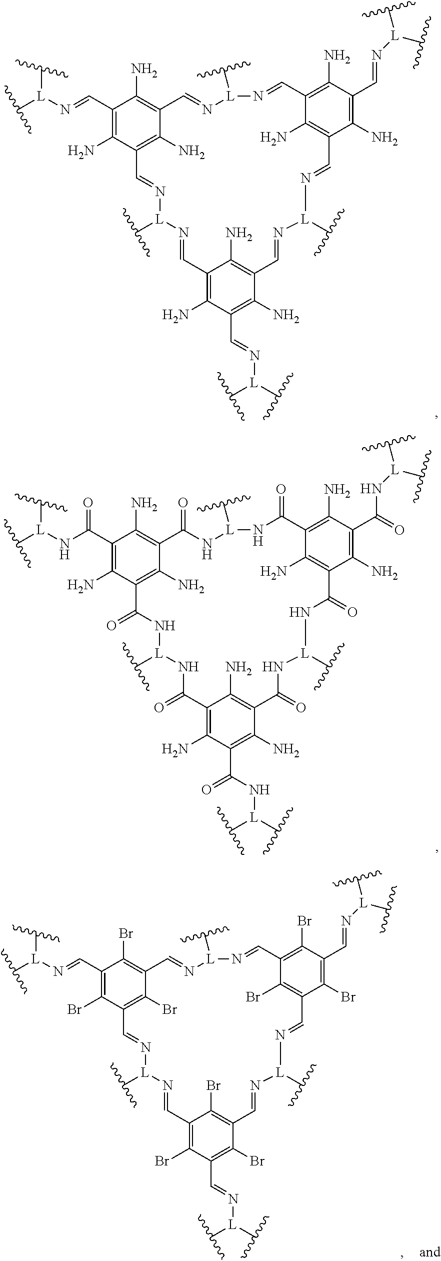

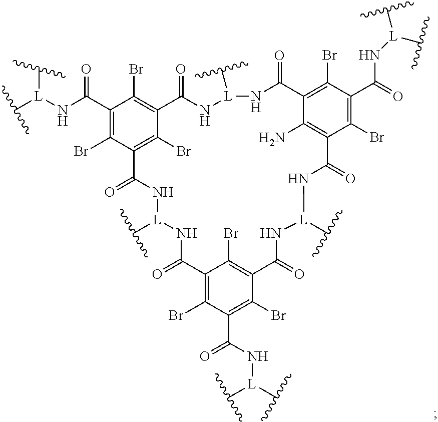

wherein each occurrence of L is

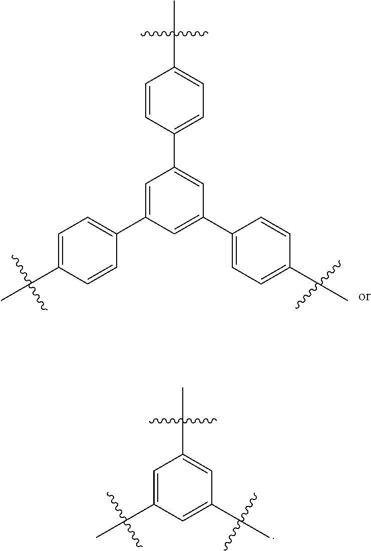 [SMILES: *c1ccc(-c2cc(-c3ccc(*)cc3)cc(-c3ccc(*)cc3)c2)cc1 ; *c1cc(*)cc(*)c1]

In certain embodiments, the polymer comprises hexagonal pores.

In other embodiments, the polymer comprises triangular pores.

In certain embodiments, the polymer is graphimine:

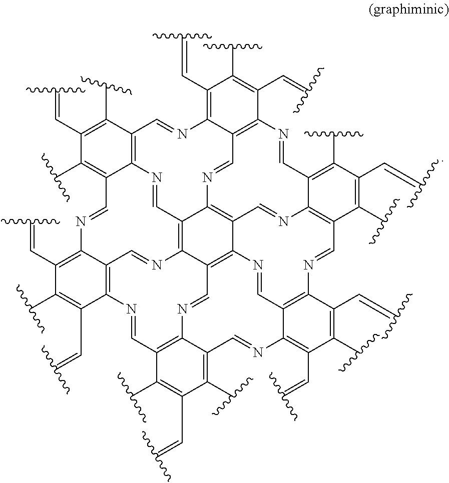 [SMILES: *c1c(C=*)c2ncc3c(*)c(C=*)c(*)c4cnc5c(C=*)c(*)c(C=*)c6ncc7c(*)c(C=*)c(*)c8cnc9c(C=*)c(*)c(C=*)c%10ncc%11c(*)c(C=*)c(*)c%12cnc(c1C=*)c2cnc1c(cnc34)c(ncc56)c(cnc78)c(ncc%109)c1cnc%12%11]

As used herein, the term “one-dimensional polymer” refers to a polymer formed from linearly or sequentially connected monomers. In certain embodiments, e.g., wherein the one-dimensional polymer is linear, the one-dimensional polymer has two end groups. By contrast, a two-dimensional polymer has an infinite number of end groups since in a 2D polymer the end groups are positioned along the edge of the 2D polymer sheet or plane. One-dimensional polymers may also be branched. In certain embodiments, branched polymers contain more than two end groups. Branched polymers may comprise secondary polymer chains attached to a main chain.

In certain embodiments, the invention provides a one-dimensional polymer formed from a plurality of hexavalent monomer units; wherein the hexavalent monomer unit is a compound of formula (I), wherein R^{B} is —NH₂ or —NH₃⁺; or

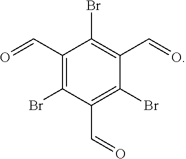

In certain embodiments, the one-dimensional polymer is linear.

In alterative embodiments, the one-dimensional polymer is branched.

The present invention also provides a copolymer, comprising a two-dimensional polymer of the invention and a one-dimensional polymer of the invention.

### Applications

The chemical arraignment of functional groups around a central benzene ring inherently provides a performance advantage, because of the aromaticity and potential for compact polymerization into high density, low mass, network work containing imine-linkages. The unique nature of the monomer units described herein allow access to novel high-performance materials in the form of covalent organic frameworks or two-dimensional polymers.

One of the current industry standards for high performance, light-weight materials is Kevlar, which is falls into the class of poly(p-phenylene terephthalamide) (PPTA) materials, as these polymer fibers are among the stiffest and strongest engineered materials per unit mass that are currently available. These PPTA fibers possess a unique combination of high stiffness and strength with resistance to elevated temperature, chemicals, ultraviolet, moisture, and creep. These characteristics are enabled by two key features of the polymer: (1) a rigid aromatic backbone that provides stiffness and strength to the molecule, and (2) strong intermolecular hydrogen bonding that provides macroscopic strength and stiffness.

The covalent organic frameworks or two-dimensional polymers formed from the fully-functionalized hexavalent benzene derivatives of the invention are expected to have even greater mechanical properties relative to Kevlar while maintaining a low mass-to-density ratio, because the monomer units allow for polymerization as extended planar sheets, instead of being limited to formation as linear polymer chains and fibers. Such planar polymers have desirable mechanical properties and the potential to become the new industry standard as a high performance, light-weight material useful for body armor, protective sheets and barriers, and protective coatings and films. Additionally, the small pores resulting from the polymer network might be useful for separations, as a selectively permeable membrane for gas or small molecule, for potential use in application such as oil refinement, gas separation and purification, liquid separation and purification, or ion separation for battery applications.

In certain embodiments, the invention provides a synthetic fiber comprising a two-dimensional polymer of the invention.

### Methods of Making

The invention also provides a method of making compound (2):

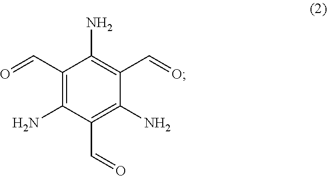 [SMILES: Nc1c(C=O)c(N)c(C=O)c(N)c1C=O]

comprising the step of combining under conditions sufficient to produce compound (2): compound (2i) and a Bronsted acid; wherein compound (2i) has the following structure:

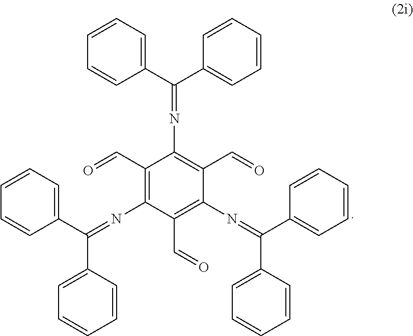 [SMILES: O=Cc1c(N=C(c2ccccc2)c2ccccc2)c(C=O)c(N=C(c2ccccc2)c2ccccc2)c(C=O)c1N=C(c1ccccc1)c1ccccc1]

In certain embodiments, the Bronsted acid is acetic acid.

In alternative embodiments, a synthetic strategy may be employed that avoids amine- or aldehyde-protecting groups. This novel approach utilizes 2,4,6-tribromobenzene-1,3,5-tricarbaldehyde (Rubin's aldehyde) as a starting material, which is then subjected to nucleophilic aromatic substitution, condensation reactions, and/or transamination, using ammonia as the nitrogen source for the formation of the novel compounds under various neat, solvate, basic, and acidic conditions. The resultant novel species have potential industrial applications since the species have the ability to become polymerized into extended planar sheets in the form of two-dimensional polymer networks.

The invention also provides a method of making a two-dimensional polymer of the invention, comprising the step of combining compound of formula (I), wherein R^{B} is —NH₂ or —NH₃⁺; or

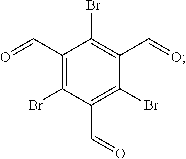 [SMILES: O=Cc1c(Br)c(C=O)c(Br)c(C=O)c1Br]

and
H₂N-L-NH₂, wherein L represents the linking moiety;
   wherein the conditions sufficient to produce the two-dimensional polymer comprise a Bronsted acid.

In certain embodiments, the Bronsted acid is acetic acid.

In certain embodiments, the invention provides a method of making any one of compounds (2)-(7), or a salt thereof, comprising the step of combining under conditions sufficient to produce any one of compounds (2)-(7):
compound (1) and a nucleophilic amine source; wherein compound (1) has the following structure:

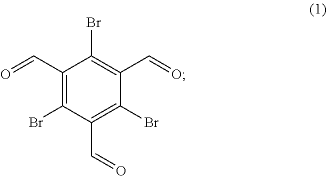 [SMILES: O=Cc1c(Br)c(C=O)c(Br)c(C=O)c1Br]

and
the amine source is selected from the group consisting of ammonia (NH₃) and salts thereof, ammonium salts, primary organic amines, and salts thereof.

In certain embodiments, the ammonium salt may be, e.g., NH₄Cl, NH₄Br, NH₄OAc, or NH₄OH.

In certain embodiments, the primary organic amine may be ethylamine, propylamine, butylamine, cyclohexylamine, benzylamine, or a salt thereof, e.g., butylammonium chloride.

In certain embodiments, the amine source is ammonia.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in chemistry and engineering are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 5%, from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any carriers, e.g., pharmaceutically acceptable carriers, and excludes other compounds.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group.

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “optionally substituted” may refer to substitution at one or more positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

The term “hydrocarbyl” as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

Salts, as used herein, include those derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

The term “hexavalent” as used herein refers to a group having six non-hydrogen substituents. For example, a hexavalent benzene is a benzene ring that is fully-functionalized; that is, substituted at each of its six ring carbons.

A Bronsted acid refers to an acid that donates a proton to a base. Exemplary Bronsted acids include hydrochloric acid, hydrobromic acid, hydroiodic acid, and oxalic acid.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the terms “weight percent,” “wt %,” or “% w/w” are meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass m₁ to the mass of the total composition m_{T} such that weight percent=(m₁/m_{T})*100.

As used herein, the terms “volume percent,” “vol %,” or “% v/v” are meant to refer to the quantity by volume of a compound and/or component in a composition as the quantity by volume of a constituent component of the composition as a percentage of the volume of the total composition.

### EXAMPLES

In order that the invention described herein may be more fully understood, the following examples are set forth. The examples described in this application are offered to illustrate the compositions and methods provided herein and are not to be construed in any way as limiting their scope.

### I. Materials and Methods

### 1. Standard Operating Procedure for Solution State Ammonia Reactions

### Experimental Description

This standard operating procedure describes the method for delivery of ammonia into a graduated Schlenk tube equipped with a Dewar-type condenser for solution state reactions. This method will detail the assembly of glassware, delivery of ammonia, operation of the condenser with dry ice, heating or cooling reaction systems, and venting the systems for reaction termination. At each step, safety and risk mitigation notes are documented for how to safely manage unexpected events.

### Required PPE and General Safety Notes

At all times the researcher is required to wear gloves, safety glasses, and flame-resistant lab coat, and in some instances additional shielding or PPE may be used at the discretion of the researcher. At no point will liquid ammonia or venting ammonia containers be used or transported outside of the chemical fume hood. At no point will the reaction set-up, active reaction, ammonia lecture bottle, or any ammonia containing solutions be near any open flames, sparks, or other source of ignition as ammonia is flammable. All operators must have completed the prerequisite lab safety training, including working with cryogenics, and have reviewed the Safety Data Sheets (SDS) for anhydrous ammonia, dry ice, and any other relevant materials before beginning work.

| Steps: | Instructions: | Safety Notes: |
|---|---|---|
| 1 | Record weight of ammonia lecture bottle. | Dropping ammonia lecture bottle has the |
| | | potential for unexpected and catastrophic |
| | | ammonia leaks. Handle lecture bottle with |
| | | great care. In the event of an unexpected |
| | | release of ammonia, close fume hood and |
| | | wait for ammonia gas to dissipate. If |
| | | lecture bottle falls out of hood onto lab |
| | | floor immediately evacuate the area. |
| 2 | Working quickly, so that glassware | All work with ammonia must be |
| | remains hot from oven (130° C.), begin | conducted within chemical fume hood. |
| | assembling reaction apparatus by first | Before beginning, ensure that there is |
| | securing the graduate two necked Schlenk | enough space for the assembly and |
| | tube with a large three fingered clamp | operation of reaction apparatus. |
| | (with stir bar - if necessary) around top of | Glassware is fragile, hang with care, avoid |
| | the graduated Schlenk flask. Attached | applying excusive pressure, dropping, and |
| | Schlenk tube to the Schlenk line using | breaking. Broken glass is sharp and has |
| | Tygon tubing and plug the remaining open | the potential for cuts or other injuries. |
| | necks with red septa. Center Schlenk tube | All clean, broken glassware is to be |
| | on stir plate with hot/cold bath if desired. | disposed of in glass waste, all |
| | | contaminated glassware is to be submitted |
| | | as solid hazardous waste. |
| | | Touching hot glassware without |
| | | protective glove will cause burns. Oven |
| | | gloves are recommended for handling of |
| | | hot glassware. |
| 3 | While glassware is still hot, open the | Needles are sharp, use carefully to void |
| | stopcock on the Schlenk tube to nitrogen | shares related injury. |
| | and purge system for three time for 10 | |
| | second interval using a needle to puncture | |
| | septa. | |
| 4 | Once the Schlenk tube has reached room | |
| | temperature, increase nitrogen flow rate | |
| | and add solids to the Schlenk tube. Reseal | |
| | with septa once addition is completed, and | |
| | decrease nitrogen purge rate. | |
| 5 | Complete reaction apparatus set-up with | |
| | the addition of the dewar-type condenser | |
| | to middle neck of the Schleck tube, | |
| | remove septa, grease joint, and secure | |
| | with clip. Then connect the top to the | |
| | dewar-type condenser to an oil bubbler, | |
| | using typing and a 14/20 hose adapter | |
| | (grease join). Work quickly so that the | |
| | glassware remains hot when it is first | |
| | flushed with nitrogen. | |
| 6 | Using gas-tight syringe dispense the | Gas-tight syringe needles are sharp. Use |
| | desired amount of solvent into the Schlenk | them carefully to avoid puncture related |
| | tube using by puncturing septa. Note the | injuries. |
| | volume of solvent on exterior of graduated | |
| | Schenk flask. Later this can be used to | |
| | approximate the amount of ammonia | |
| | delivered to the reaction system. | |
| 7 | Dissolve solids. | |
| 8 | Once the solids have been satisfactorily | |
| | dissolved in the solvent. Increase nitrogen | |
| | pressure and replace septa with once that | |
| | has been puncture with HDPE tubing and | |
| | adapter to ammonia bottle. Purge line with | |
| | nitrogen for 10 second then attach to | |
| | ammonia lecture bottle. Make sure 180- | |
| | CGA adapter contains Teflon O-ring. | |
| | While system is under increased pressure | |
| | nitrogen, listen for leaks. If leaks are | |
| | detected, tighten joints. Decrease nitrogen | |
| | pressure once attachment is complete to a | |
| | slow bubbling rate. Do not open ammonia | |
| | lecture bottle at this step! | |
| 9 | Bring solvent to desired temperature. | Solubility of ammonia decreases with |
| | | increase in temperature. If a temperature |
| | | change from cold to warm is required, be |
| | | prepared for venting of ammonia gas, as |
| | | the solubility of ammonia decrease. |
| | | Rapid warming has the potential for rapid |
| | | evolution of ammonia gas, ensure that oil |
| | | bubbler vent is place toward back of fume |
| | | hood. |
| 10 | Once solvent has held the desired | Skin contact with dry ice will cause burns, |
| | temperature for a few minutes, fill the | wear protective gloves. |
| | dewar-type condenser with dry ice. | Dry ice produces carbon dioxide gas |
| | | through sublimation. Store and use in well |
| | | ventilate area to avoid asphyxiation. |
| 11 | Close stopcock on Schlenk tube. | |
| 12 | Slowly open primary ammonia lecture | If any unintentional leaks within reaction |
| | bottle value with a ½ turn. Then slowly | systems are detected, immediately close |
| | open secondary valve until bubble is | ammonia lecture bottle and close fume |
| | visualized within solution of Schlenk tube | hood sash until all ammonia has |
| | (bubble rate should be slow). Also look | dissipated. |
| | for bubbling of oil bubble. | Use of additional shielding is |
| | | recommended in the event of minor or |
| | | catastrophic failure of the reactor due to |
| | | leaks, unexpected permanent gas |
| | | production, overheating, thermal shock, |
| | | incorrect assembly, sudden and |
| | | unexpected release of ammonia, fire, and |
| | | catastrophic failure of vessel with ejection |
| | | of glass and liquid/vapor into the working |
| | | area. At no point is the Schleck tube or |
| | | condenser to leave the hood space while |
| | | ammonia is actively being used. |
| | | Before conducting reaction, operator |
| | | should prepare for the unexpected |
| | | pressure increases due to overheating, or |
| | | through production of permanent gases |
| | | during reaction, but examining reaction |
| | | conditions for any possible permanent gas |
| | | production. |
| 13 | Fill apparatuses with ammonia until drips | |
| | of ammonia can be visualized on the | |
| | interior walls of the condenser. Then close | |
| | lecture bottle and secondary ammonia | |
| | valves. Note any changes in volume from | |
| | reading the graduations on the exterior of | |
| | Schleck tube after the delivery of | |
| | ammonia. The maximum amount of | |
| | ammonia capable of saturating the | |
| | reaction solvent at the reaction | |
| | temperature should be estimated from | |
| | literature before conducting any | |
| | experiments. | |
| 14 | Let reaction run, while monitoring dry ice | |
| | levels within the condenser and amount of | |
| | ammonia on condenser. After a few hours, | |
| | and ammonia has been consumed, in may | |
| | be necessary to repeat steps 12-13 for | |
| | addition of more ammonia. | |
| 15 | Once reaction has completed, all excess | |
| | ammonia is to be vented through oil | |
| | bubbler from reaction system. Remove | |
| | dry ice from condenser and allow reaction | |
| | solution to reach room temperature. | |
| 16 | Collect product using organic synthetic | All hazardous materials need to be |
| | methods. Re-weight ammonia lecture | properly disposed of as hazardous waste. |
| | bottle to determine amount of ammonia | |
| | delivered to reaction | |

### 2. Standard Operating Procedure for Ammonia Gas Manifold and Parr Reactor

### Experimental Description

This standard operating procedure describes the method for delivery of ammonia to a Parr reactor for chemical synthesis. This method will detail the preparation of the gas manifold for ammonia delivery, delivery of ammonia, condensing of liquid ammonia within the Parr reactor, sealing off the reactor, heating for duration of experiment, venting of reactor systems in preparation of opening reactor, and cleaning rector system once the experiment has been conducted. At each step safety and risk mitigation notes are documented for how to safety deal with unexpected events. Leaking and pressure testing is conducted once every 3 months, or any time changes are made to the gas manifold, or if it has been moved. The leak and pressure testing protocol is described in a separate section from the method of operation.

### Required PPE and General Safety Notes

At all times researcher is required to wear gloves, safety glasses at all times, and in some instances additional shielding or PPE may be used at the discretion of the researcher. At no point will liquid ammonia or venting ammonia containers be used or transported outside of the fume hood. At no point will the gas manifold, Parr reactor, or ammonia lecture bottle be near any open flames, sparks, or other source of ignition as ammonia is flammable. All operators must have completed the prerequisite lab safety training, including working with cryogenics, and have reviewed the Safety Data Sheet (SDS) for anhydrous ammonia, before beginning work.

### A. Method of Operation

| Steps: | Instructions: | Safety Notes: |
|---|---|---|
| 1 | Ensure gas manifold is securely fastened | The Parr reactor body is heavy and gas |
| | to scaffold or ring stands within fume | manifold is awkward to hold with |
| | hood. Gas manifold needs to be in an | protruding ends. In the event of moving |
| | upright position for successful delivery of | the gas reactor, wear eye-protection to |
| | ammonia to reactor system. | avoid accidental eye injuries with |
| | | manifold ends. Make sure clamps are |
| | | secure to avoid toppling of reactor and gas |
| | | manifold, which could potentially cause |
| | | injuries or break fume hood glass. |
| 2 | Once manifold is secured, attach | Parr reactor assembly and tightening |
| | assembled Parr reactor to systems and | process is described in the Parr 4740 |
| | secure top of the Parr reactor head with a | manual. |
| | clamp. There should be at least 8 inches of | |
| | space between end of the reactor body and | |
| | bench top, for a small liquid nitrogen | |
| | dewar and lab jack to fit. | |
| | Secure thermal couple to exterior of Parr | |
| | reactor body | |
| 3 | Record the initial mass of the ammonia | Dropping ammonia lecture bottle has the |
| | lecture bottle. Then secure ammonia | potential for unexpected and catastrophic |
| | lecture bottle to ring stand and position to | ammonia leaks. Hand lecture bottle with |
| | attach to gas manifold. Do not open | great care. In the event of an unexpected |
| | ammonia at this point! | release close fume hood and wait for |
| | | ammonia gas to dissipate. If lecture bottle |
| | | falls out of hood, on to lab floor |
| | | immediately evacuate the area. |
| 4 | Attach ammonia lecture bottle to gas | If PTFE O-ring is missing systems will |
| | manifold by securing CGA-180 to ⅛ in | likely leak. |
| | male NPT gas regulator inlet adaptor. | |
| | Ensure that PTFE O-ring is in place. | |
| | Tighten using small wrench, until joint is | |
| | secure, but not over tightened. Do not | |
| | open ammonia at this point! | |
| 5 | Attach primary release valve to vacuum | |
| | and secondary release valve to nitrogen | |
| | source using Tygon tubing. | |
| 6 | Purge gas manifold of air and moisture, by | Do not open ammonia lecture bottle at this |
| | cycling system between nitrogen and | point! |
| | vacuum. To purge the whole systems all | During cycling, if hissing at joints is |
| | valve, except ammonia lecture bottle top, | heard, stop reaction set-up and change to |
| | should be opened. The primary and | leak testing protocol. |
| | secondary valves will alternate from their | Do not heat/melt plastic knobs or pressure |
| | open and closed positions depending on | gauge. |
| | which point you are in the cycling. Begin | |
| | by pulling vacuum for 5 minutes (vacuum | |
| | source is open, primary release valve is | |
| | open, but secondary release valve is | |
| | closed), then closing primary release | |
| | valve, filling with nitrogen for 1 minute | |
| | until approximately 15 psi is achieved by | |
| | opening nitrogen source and secondary | |
| | release valve, close secondary release | |
| | valve (to nitrogen), re-open primary | |
| | release valve (to vacuum source), and | |
| | leave system under vacuum for 10 | |
| | minutes. Repeat this process three times, | |
| | at which point systems has been fully | |
| | purged of air and residual moisture. | |
| | Heat gun | |
| 7 | Once gas manifold has been fully cycled, | |
| | close vacuum source and primary release | |
| | valve, leaving manifold and Parr reactor | |
| | under static vacuum. Double check that | |
| | both primary and secondary release valves | |
| | are sealed. | |
| 8 | Close secondary ammonia valve. | |
| 9 | Open lecture bottle valve with ⅛ turn. | While opening ammonia lecture bottle |
| | Then open secondary ammonia valve with | and delivering ammonia to system, |
| | ⅛ turn. Watch pressure gauge for any | operator is recommended to wear elbow |
| | changes in pressure. Slow open lecture | length butyl ruble glove, well fitting |
| | bottle with ½ turn, open secondary | (splash resistant) goggles, and flame- |
| | ammonia valve with ½ turn. Continue in | resistant lab coat. Use of additional |
| | this manner while carefully watching | shielding (portable blast shield) around |
| | pressure gauge for any pressure changes. | Parr is recommended in the event of a |
| | Once a pressure change is detected, allow | catastrophic failure. |
| | whole system to pressurize (wait 3-5 | If a small leak is detected, close ammonia |
| | minutes), then open secondary ammonia | bottle, then close fume hood while |
| | valve fully (3 full turns), and ammonia | ammonia dissipates. |
| | lecture bottle fully (3 full turns). | If a catastrophic leak (or break) is |
| | Record reading on pressure gauge. | detected. Close fume hood and wait for |
| | | ammonia lecture bottle to empty and |
| | | fumes to dissipate. |
| 10 | Once stabile pressure has been reached, | Liquid nitrogen can cause burns, operator |
| | the operator can begin condensing liquid | must have completed cryogenic safety |
| | ammonia within Parr reactor. Using a lab | training before use, be wearing lab coat, |
| | jack and small liquid nitrogen dewar, | goggles, and gloves, and avoid |
| | submerge bottom ⅓ of Parr reactor in | spilling/splashing liquid nitrogen |
| | liquid nitrogen. Wait 30 minutes, while | whenever possible. |
| | continual topping of liquid nitrogen in | The temperature shock of the submerging |
| | dewar. After 30 minutes, submerge the | the Parr reactor body within liquid |
| | next ⅓, wait another 30 minutes, then | nitrogen, can cause catastrophic failure of |
| | submerge the final ⅓ of the reactor. | the rector. Additional shielding is |
| | | recommended. Apparatus is not to leave |
| | | fume hood. |
| 11 | Once fully submerged in liquid nitrogen, | |
| | wait an additional 30 minutes, for the | |
| | ammonia to condense within the reactor | |
| | body. | |
| | Record reading on pressure gauge. | |
| 12 | Then close ammonia lecture bottle and | |
| | secondary ammonia valve. | |
| 13 | Removed liquid nitrogen dewar and allow | |
| | Parr reactor to warm to room temperature | |
| | (1 h). | |
| | Record reading on pressure gauge. | |
| 14 | Once reactor has reached room | The maximum possible pressure of the |
| | temperature, oil bath and stir plate can be | reactor systems is 1000 psi, or 68.94 bar, |
| | used if desired. | which is dictated by the pressure capacity |
| | Add heating method in detail. | of the needle valve that closes the Parr |
| | Record reading on pressure gauge at new | reactor. The maximum recommended |
| | temperature. | operating pressure is 19.32 bar or 280.24 |
| | | psi, which is only produced when the |
| | | ammonia is heat to 50° C. Pressure testing |
| | | of the system are conducted to 600 psi. |
| | | Use of additional shielding is |
| | | recommended in the event of minor or |
| | | catastrophic failure of the reactor due to |
| | | leaks, unexpected permanent gas |
| | | production, overheating, thermal shock, |
| | | incorrect assembly, sudden and |
| | | unexpected release of ammonia, fire, and |
| | | catastrophic failure of vessel with ejection |
| | | of metal and liquid/vapor into the working |
| | | area. At no point is the reactor or gas |
| | | manifold to leave the hood space while |
| | | ammonia is actively being used. |
| | | Before conducting reaction, operator |
| | | should prepare for the unexpected |
| | | pressure increases due to overheating, or |
| | | through production of permanent gases |
| | | during reaction, but examining reaction |
| | | conditions for any possible permanent gas |
| | | production. |
| | | In the event of unexpected pressure |
| | | increase, open secondary release value. |
| 15 | The sealed reactor and corresponding | In case of slow depressurization of Parr |
| | valves are to remain sealed for the | reactor over length of reaction, Parr |
| | duration of reaction time (1 h to 5 days). | reactor is to remain in fume hood, with |
| | | sash closed. |
| 16 | Once reaction has competed, the reactor | Thermal shock of vessels can lead to |
| | body is to slowly cooled (or warmed) to | catastrophic failure and ejection of metal |
| | room temperature, by removing oil bath | and liquid/vapor into the working area |
| | (wait 1 h - after heat/cooling source has | |
| | been removed). | |
| 17 | Then slowly open reactor valve, and | |
| | record pressure. | |
| 18 | Depressurize the reactor and gas | Exposure to ammonia gas is toxic and case |
| | manifold, by slowly opening secondary | burns. Recommend PPE includes elbow |
| | release valve. Tubing for secondary | length butyl rubber glove, lab coat, splash |
| | release valve is used to direct ammonia | goggle. Hood is only to be opened a |
| | gas to hood vent any away from operator | minimal amount of allow to access to |
| | and open hood sash. Once valve is cracked | valve during depressurization. |
| | open (first ¼ turn) and depressurization | In the event that depressurization occurs at |
| | has begun, wait 10 minutes for system to | a rate more rapid than expect, hood is to |
| | fully depressurize, then open secondary | be close immediately, and remain close |
| | release valve fully and wait another 5 | unit all ammonia gas has been expunged. |
| | minutes. | |
| 19 | Once initial de-pressurization has been | |
| | complete open both primary and | |
| | secondary release valves (at this point | |
| | systems should no longer be pressurized). | |
| | Allow valves to remain open for 10 | |
| | minutes. | |
| 20 | Flush gas manifold with nitrogen by | |
| | connecting primary release valves to | |
| | nitrogen source and flowing small stream | |
| | of nitrogen through systems for 10 | |
| | minutes. | |
| 21 | Once system has been flushed with | Wearing nitrogen gloves, lab coat, and |
| | nitrogen, the reactor is safe to | eyeglasses is recommended when |
| | disconnected and disassembly. | handling reactor system. |
| 22 | Removed products vial for analysis. Wash | All hazardous materials need to be |
| | reactor body and parts in water to remove | properly disposed of as hazardous waste. |
| | any residue ammonia. Dry parts | |
| | completely once clean. | |
| 23 | Disconnect ammonia lecture bottle, | |
| | record its new mass, and place in storage | |
| | area. | |
| 24 | Visually inspect gas manifold for any | |
| | damages, scratches, or other changes that | |
| | may have occurred during assembly, | |
| | reaction, or disassembly. | |

### B. Leak and Pressure Testing Protocol

Pressure test will be conducted using nitrogen gas cylinder. The pressure release valve is connected to a nitrogen cylinder. The pressure is incrementally increased by 50 psi using a regulator on the nitrogen cylinder. At each increment, all joints, valves, connections, and the top of the Parr reactor is applied with a soapy solution. If a leak is present, bubble will be produced at leak sight. If no bubbles are observed reactor is sealed and left in pressurized state overnight. Reaction pressure is checked overnight to determine quality of seals around reactor. Pressure test is conducted up to 600 psi, more than double the highest operating pressure.

| Steps: | Instructions: | Safety: |
|---|---|---|
| 1 | Fully assemble gas manifold and Parr | |
| | reactor, include attachment of ammonia | |
| | lecture bottle. Do not open ammonia | |
| | lecture bottle valve. | |
| 2 | Connect secondary release valve to | |
| | nitrogen gas cylinder equipped with a | |
| | regulator. Make sure connection is | |
| | secure, utilize hose clamps as necessary. | |
| 3 | Incrementally increased by 50 psi using a | Use of additional shielding is |
| | regulator on the nitrogen cylinder. Check | recommended in the event of minor or |
| | pressure read-out on gas manifold gauge. | catastrophic failure of the reactor due to |
| | | leaks, over pressurization, incorrect |
| | | assembly, and catastrophic failure of |
| | | vessel with ejection of metal into the |
| | | working area. At no point is the reactor or |
| | | gas manifold to leave the hood space |
| | | while being pressurized. |
| 4 | At each increment, all joints, valves, | |
| | connections, and the top of the Parr | |
| | reactor is applied with a soapy solution. If | |
| | a leak is present, bubble will be produced | |
| | at leak sight. If leak is present, re-tighten | |
| | joint, and re-evaluate. | |
| 5 | Once pressure has reached 600 psi and no | |
| | leaks are present. Rector top valve is | |
| | sealed for overnight pressure test. Once | |
| | top vale is scaled, nitrogen source and all | |
| | other valves are closes. | |
| 6 | The pressurized Parr reactor is left | |
| | overnight. | |
| 7 | The next day the top valve is opened to | |
| | measure the system's pressure. If pressure | |
| | has not changed, the system is good for | |
| | reactions, if pressure has decreased the | |
| | reactor is tightened, re-evaluated, and | |
| | pressure testing protocol is repeated. | |
| 8 | Once pressure has been completed | |
| | satisfactorily, system is depressurized by | |
| | slowly opening primary release valve, and | |
| | ready for reactions. | |

### II. Procedures

### Example 1. Conversion of Rubin's Aldehyde to 2,4,6-triaminobenzene-1,3,5-tricarbaldehyde

### Method A.

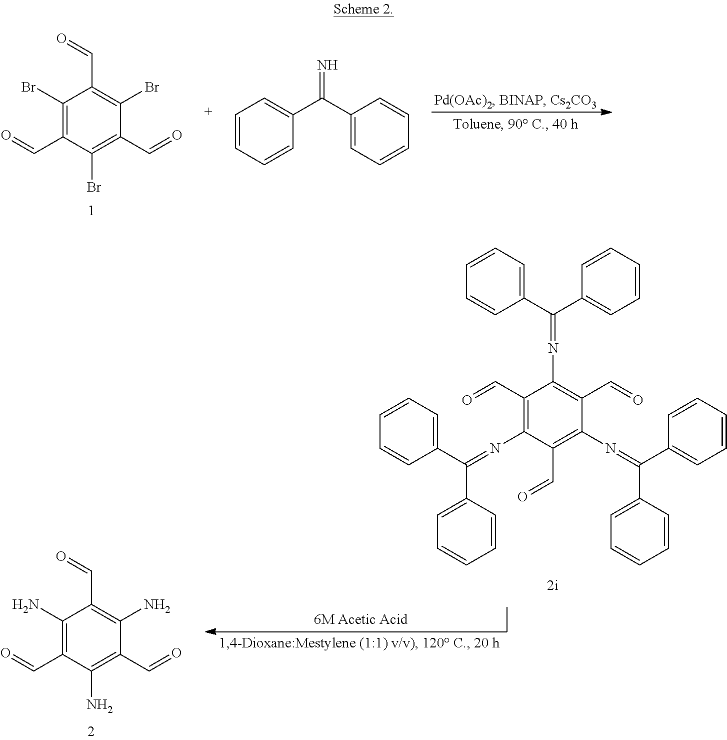 [SMILES: O=Cc1c(Br)c(C=O)c(Br)c(C=O)c1Br ; N=C(c1ccccc1)c1ccccc1 ; Nc1c(C=O)c(N)c(C=O)c(N)c1C=O ; O=Cc1c(N=C(c2ccccc2)c2ccccc2)c(C=O)c(N=C(c2ccccc2)c2ccccc2)c(C=O)c1N=C(c1ccccc1)c1ccccc1]

### Preparation of 2i from Buchwald-Hartwig Coupling with 1 and Benzophenone Imine, Followed by Acid Catalyzed Deprotection with Acetic Acid for the Formation of 2

Scheme 2 depicts the design of novel monomer 1,3,5-tribenzopheonoeimine-2,4,6-tricarboxyaldehyde (2i). This monomer contains alternative aldehyde and benzophenone imines functionalities. The advantage of using benzophenone imine as both the protecting group and amine source is that it can undergo acid-catalyzed deprotection for the formation of 2 in Scheme 2, allowing for further chemistry using these novel hexasubstituted benzenes. The synthesis of 2 begins with the formation of Rubin's aldehyde (1), from 1,3,5-tribromobenzene with a Friedel-Craft Alkylation to form 1,3,5-tribromo-2,4,6-tris(dichloromethyl)benzene, then a subsequent hydrolysis. Then Buchwald-Hartwig coupling is conducted with benzophenone imine, for the substitution of benzophenone imines at the aryl-bromides resulting in the successful synthesis of 2, after purification.

Compound 2i was prepared from 1 using the Buchwald-Hartwig coupling described below and depicted in Scheme 2. The structure of 2i was confirmed with ¹H and ¹³C NMR. Compound 2i ¹H NMR (CD₂Cl₂) 9.95, 7.47, 7.45, 7.43, 7.31, 7.29, 7.27, 7.25, 7.23, 5.30, 1.52); ¹³C NMR (CDCl₃) 187.94, 169.1, 159.02, 136.93, 130.64, 129.23, 128.44, 108.53, 77.22.

Additionally, the molecular weight was confirmed using matrix assisted laser desorption/ionization-time of flight mass spectrometry (MALDI-TOF-MS), with an ionization peak of 700 m/z, and a melting point of 271° C. measured using differential scanning calorimetry. The deprotection of 2i, through the addition of a 6 M acetic acid solution, resulted in the formation of 2 and benzophenone, which was also confirmed using ¹H NMR.

Rubin's aldehyde, 1, a previously reported compound, can be efficiently synthesized from a two-step process beginning with the Friedel-Crafts Alkylation of 1,3,5-tribromobenzene in chloroform, to form 1,3,5-tribromo-2,4,6-tris(dichloromethyl)benzene, which is then hydrolyzed in concentrated sulfuric acid with FeSO₄, for the efficient production of 1.⁵ The Buchwald-Hartwig coupling was conducted with palladium (II) acetate (0.73 g, 3.26 mmol) and rac-BINAP (2.54 g, 4.07 mmol) in 50 mL of anhydrous toluene and activated at 50° C. for 15 minutes, until magenta color persisted. Then 1 (6.50 g, 16.3 mmol), Cs₂CO₃ (26.6 g, 81.5 mmol), and benzophenone imine (10.3 g, 57.0 mmol) were added with an additional 100 mL of anhydrous toluene. Once combined, the flask was sealed with a Teflon cap and placed in at pre-heated oil bath at 90° C. for 40 h. The solution was evacuated to dryness, and dissolved in dichloromethane, which was then extracted twice with 100 mL of water. The organic phase was then concentrated and dissolved in a minimal amount of ethyl acetate, and chilled to 0° C., to encourage the precipitation of 2i, which was collected using vacuum filtration. The crude product was then further purified with flash chromatography using a mobile phase gradient hexane:dichloromethane (30:70 v/v)→100% dichloromethane, for isolation of 2i as a light-yellow powder, 1.89 g or 16.6%.

Compound 2 resulted from the deprotection of 2i with 6 M acetic acid in a 1,4-Dioxane:Mestylene (1:1, v/v) solution, at 120° C. for 20 h, which produced benzophenone and 2 as a brown precipitate. Notably, this method successful provided new compound 2, and evidence that it is a stable. It was hypothesized that this compound may not be stable as it contains adjacent reactive functional groups within close proximity. Further efforts were dedicated to making compound 2 with higher yield.

Compound 2 ¹H NMR (DMSO-d₆ at 25° C., with a 500 MHz NMR): 9.95, 9.04, 7.29, 7.03, 2.50 (trace ammonia at 7.16 ppm, acetone at 2.08 ppm, and water at 3.38 ppm). ¹³C NMR (DMSO-d₆ at 25° C. with a 400 MHz NMR): 185.99, 160.58, 95.64, 39.50. ¹H NMR (CDCl₃): 9.94, 9.02, 3.34, 2.50).

### Example 2. Conversion of Rubin's Aldehyde to 2,4,6-triaminobenzene-1,3,5-tricarbaldehyde

### Method B.

Compound 2 may alternatively be accessed through nucleophilic aromatic substitution (SnAr) with Rubin's aldehyde and ammonia as shown in Scheme 3 below.

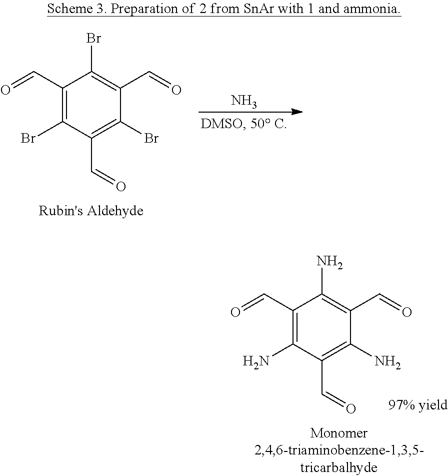

This nucleophilic aromatic substitution reaction using Rubin's aldehyde and ammonia produced a much shorter, higher yielding (97%), and more efficient route than through the intermediacy of 2i. Both ¹H and ¹³C NMR spectra confirmed the structure of 2, and therefore successful synthesis of the monomer species via SnAr with ammonia, as a shorter, simpler, cost and time effective method for producing 2.

The solution state synthesis of 2,4,6-triaminobenzene-1,3,5-tricarbaldehyde (2), is done through nucleophilic aromatic substitution of the bromines occupying the 2, 4, and 6 positions on 2,4,6-tribromobenzene-1,3,5-tricarbaldehyde (1), a previously reported compound. Using a two necked graduated Schlenk tube containing a nitrogen atmosphere, 1 is dissolved in a solution of anhydrous dimethyl sulfoxide (DMSO) (40 mL per 1 g of 1). The solution, equipped with a magnetic stir bar and 3 Å molecular sieves, is heated to 50° C. The top of the reactor is connected to a dewar-type condenser containing a cold bath at a temperature of at least −33° C. (the point at which ammonia condenses to a liquid state), and the dewar-type condenser outlet is connected to an oil bubbler to prevent pressure build-up. Anhydrous ammonia gas is bubbled through the DMSO solution, at a rate of 0.75 g of ammonia per one hour, this persists for a period of at least one hour until a brown color is observed. When the reaction is completed the ammonia gas valve is closed, the system is purged with nitrogen gas for a period of 10 minutes, and the reaction solution is vacuum filtered to collect the molecular sieves. The solution is then reduced under high vacuum at 50° C. for a period of 3 hours, resulting in the formation of 2 as a brown precipitate, which is then collected using vacuum filtration. Compounds 3, 4, 5, 6, and 7, can be synthesized in a similar matter.

Additionally, through the use of liquid ammonia, compounds 2, 3, 4, 5, 6, and 7, can be synthesized. For delivery of liquid ammonia to a Parr 4740 reactor a gas manifold has been constructed that contains, an ammonia source, a primary ammonia control valve (or lecture bottle valve or ammonia gas regulator), a secondary ammonia control valve, a primary release valve, a pressure gauge, a reactor valve, and a secondary release valve. During the assembly of the reactor body, 1 is placed within a Teflon liner or glass vial, in addition to any additional reagents that are required for the reaction. The ammonia is delivered through the gas manifold to the Parr 4740 reactor, which has been put under negative pressure (static vacuum). The primary ammonia control valve, secondary ammonia control valve, and reactor valve are open, allowing the ammonia to fill and pressurize the reactor. The reactor body is then placed in liquid nitrogen to condense and fill the reactor with liquid ammonia. Once the reactor body is filled the primary ammonia control valve, secondary ammonia control valve, and reactor valve are closed. The reaction is then allowed to go for a period of 30 mins to five days, at which point the pressure is released and products are isolated.

The reactivity of compound 2 was studied to compare the reactivity of the amino groups versus the aldehyde groups in formation of imines (Scheme 3A).

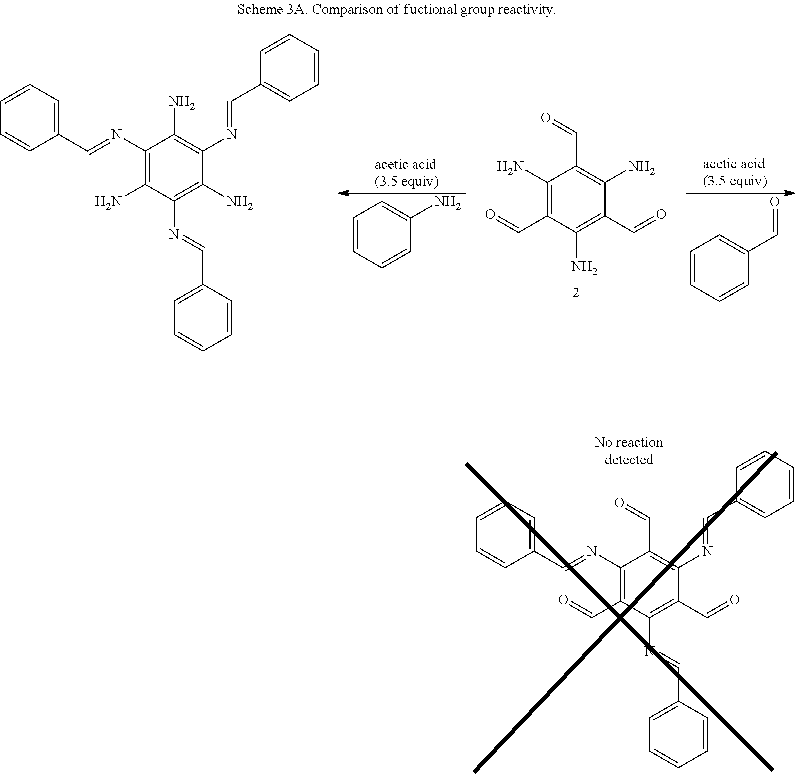

By isolating and simplifying each reactive group, it was determined that the amino groups, which are typically classified as good or moderate nucleophiles, are poor nucleophiles in compound 2. The amino groups therefore do not initiate the first step in the imine-condensation reaction mechanism, inhibiting polymerization into graphimine according to the methods used for detection.

This reactivity challenge can be attenuated by altering the electronic structure of the benzene ring through the removal of the aldehydes, which are strong electron withdrawing groups, therefore altering the amino-group reactivity.

### Example 3. Production of Graphimine from 2,4,6-triaminobenzene-1,3,5-tricarbaldehyde

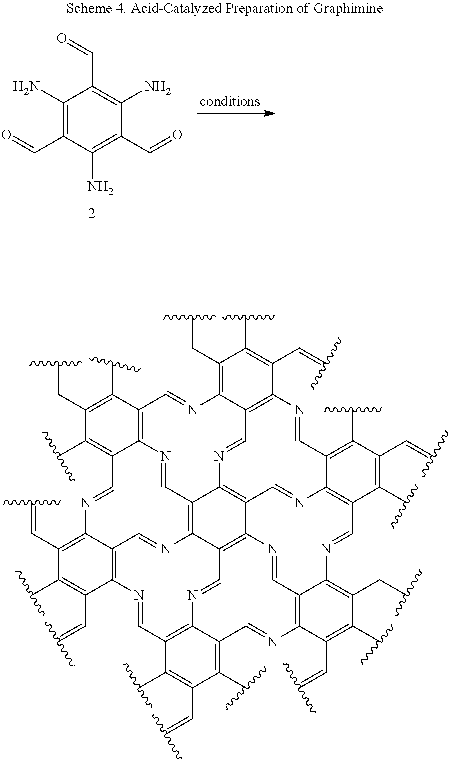 [SMILES: Nc1c(C=O)c(N)c(C=O)c(N)c1C=O ; *Cc1c(*)c2cnc3c(C=*)c(*)c(C=*)c4ncc5c(*)c(C=*)c(*)c6cnc7c(C=*)c(*)c(C*)c8ncc9c(*)c(C=*)c(*)c%10cnc%11c(C=*)c(*)c(C*)c%12ncc(c1*)c2ncc1c(ncc43)c(cnc56)c(ncc78)c(cnc%109)c1ncc%11%12]

### Overview of Reaction Conditions and Results

| Acid | Monomer | Conditions | Results |
|---|---|---|---|
| TFA (100%) | 2 | 60° | No reaction |
| | | | detected |
| 95:5 (v/v) | 2 | 50° | No reaction |
| TFA:Water | | | detected |
| 2M Acetic Acid/ | 2 | 90°, DMSO/ | No reaction |
| 3.6M Water | | water | detected |
| 20 Eqv. Acetic | 2 | 120°, anhydrous | Precipitate, |
| Acid | | DMSO | 41% yield |
| 10 Eqv. pTSA | 2 | 70°, 6:4 (v/v) 2- | Precipitate, |
| | | propanol:DMSO | 47% yield |
| 3M Acetic Acid | 2 + 1,4- | 70° | Precipitate, |
| | phenylenediamine | | 90% yield |

As shown in the table above, two conditions produced promising graphimine materials (and one related COF)
The following are indications of a successful polymerization:
1. Formation of a solid precipitated from the reaction solutions
2. Upon collection, precipitate should be insoluble in all organic solvents
3. FTIR analysis should indicant a reduction in amino- and aldehyde groups, as well has the formation of imine linkages

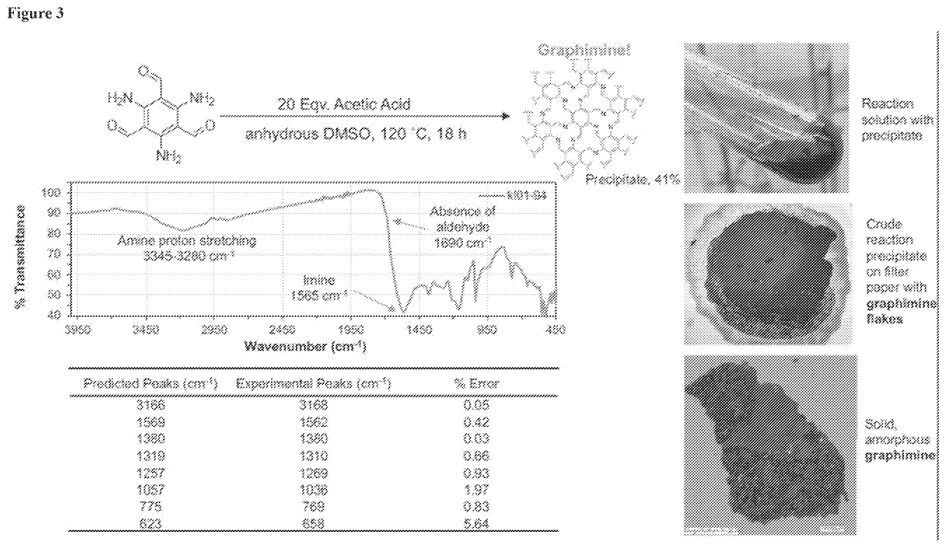

Results of the polymerization catalyzed by 20 equivalents of acetic acid are shown in FIG. 3. From this reaction, the resulting precipitate was insoluble in all organic solvents, except TFA, which was believed to have degraded resulting polymer.

FTIR analysis confirmed formation of imine bonds, lack of aldehydes, and some residual amino-groups; and is comparable to the computationally predicted graphimine FTIR spectrum.
Resulting material is amorphous (lacks any crystallinity), as determined from PXRD.
Graphimine was produced in 41% yield.

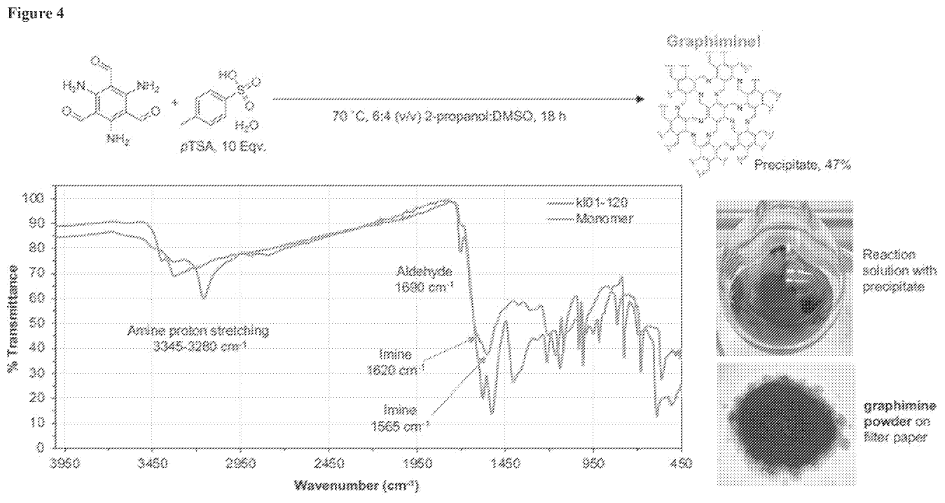

Results of the polymerization catalyzed by 10 equivalents of para-toluenesulfonic acid are shown in FIG. 4. From this reaction, the resulting precipitate was insoluble in all organic solvents, except TFA, which was again believed to have degraded resulting polymer. Additionally, the resulting material is amorphous (lacks any crystallinity—determined from PXRD).

FTIR analysis is consistent with predicted graphimine spectrum, the spectrum above confirmed formation of imine bonds, reduction of aldehydes, and amino-groups Comparison to the monomer's FTIR spectrum reveals additional differences within the fingerprint region (below 1500 cm⁻¹), and large reductions in free amino- and aldehyde groups.
Graphimine was produced in 47% yield.

### Example 4. Production of a 1,4-diphenyl-imine-linked COF from 2,4,6-triaminobenzene-1,3,5-tricarbaldehyde

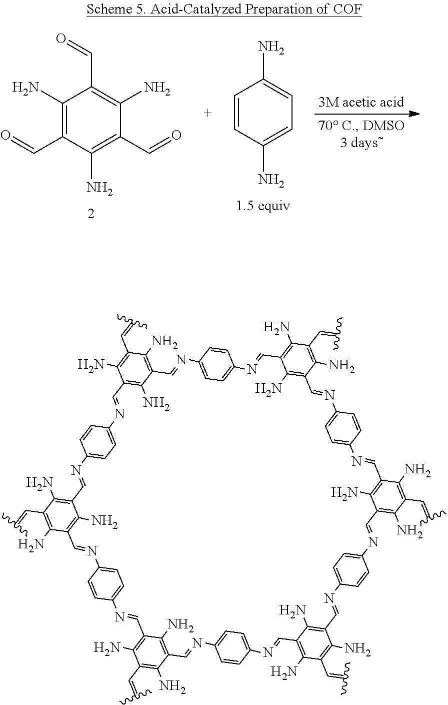 [SMILES: Nc1c(C=O)c(N)c(C=O)c(N)c1C=O ; Nc1ccc(N)cc1 ; *=Cc1c(N)c2cnc3ccc(cc3)ncc3c(N)c(C=*)c(N)c(cnc4ccc(cc4)ncc4c(N)c(C=*)c(N)c(cnc5ccc(cc5)ncc5c(N)c(C=*)c(N)c(cnc6ccc(cc6)ncc6c(N)c(C=*)c(N)c(cnc7ccc(cc7)ncc7c(N)c(C=*)c(N)c(cnc8ccc(cc8)ncc(c1N)c2N)c7N)c6N)c5N)c4N)c3N]

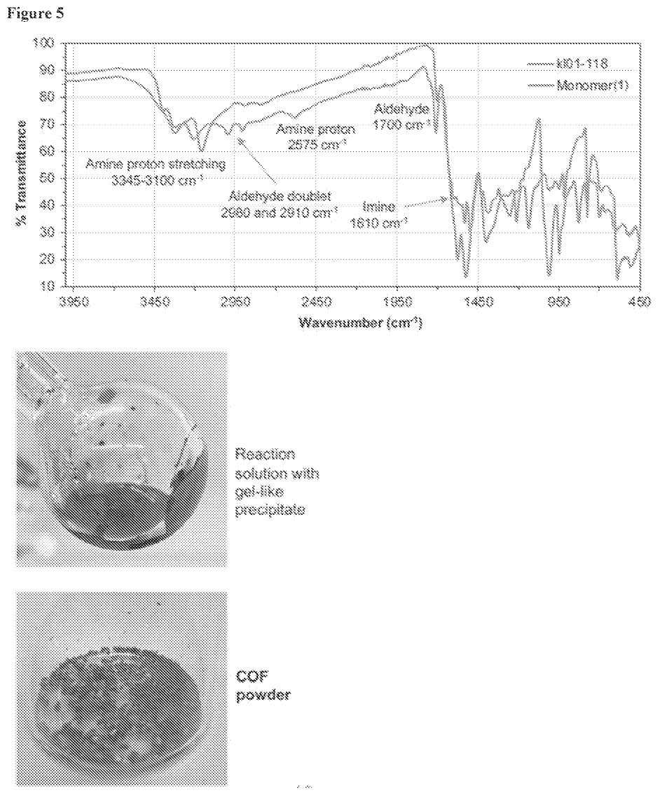

Results of the polymerization of compound 2 with 1,4-phenylenediamine catalyzed by 3M acetic acid are shown in FIG. 5. The product was produced in 90% yield.

FTIR analysis confirmed formation of imine bonds and retention of amino group. However, there was still a large excess of aldehyde groups, which can be attributed to suspension of unreacted aldehyde groups within polymer matrix.

This material is also insoluble in all organic solvents and completely amorphous (as determined from PXRD).

### Example 5. Alternative Route to Graphimine from 2,4,6-triaminobenzene-1,3,5-tricarbaldehyde

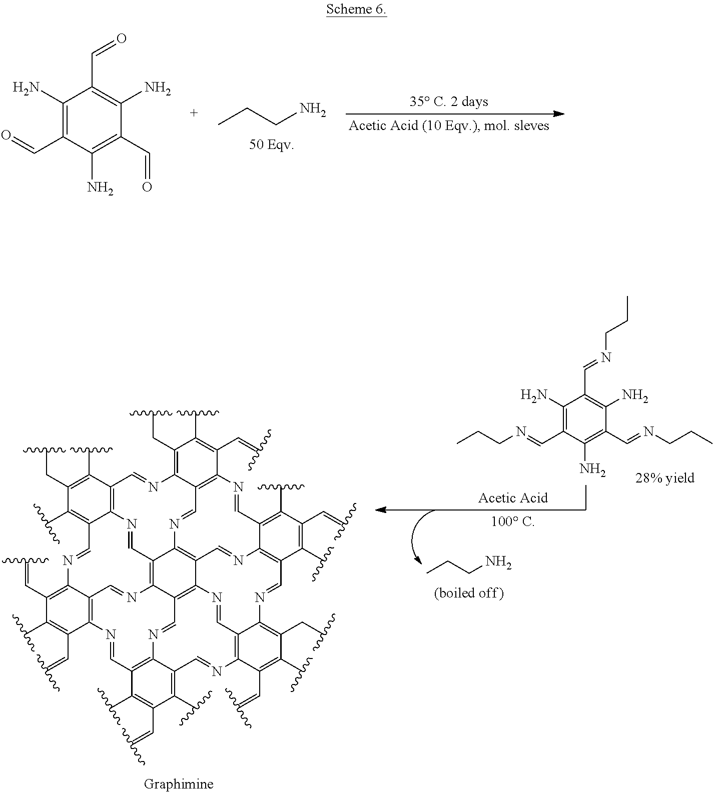

### Graphimine Via Propylene-Functionalization of Compound 2

Amines, such as aniline, readily reacts with the aldehyde functionalities on the 2,4,6-triaminobenzene-1,3,5-tricarbaldehyde.

Thus, the propylamine-functionalized monomer, with altered amino-reactivity, may be used in the synthesis of graphimine, and the propylamine can be easily driven from the reaction solution by taking advantage of its low boiling point (47.8° C.).

The propylamine-functionalized monomer was subjected to polymerization conditions with 3 equivalents acetic acid at 100° C. over 2 days in 1:1 (v/v) dioxane/mesitylene. After the first 18 h a gel-like precipitate had formed at the bottom of the reaction flask. ¹H NMR revealed evidence of intermonomer imine-linkages, suggesting that some degree of polymerization has occurred. It is likely that once polymerized, even at an oligomeric level, the oligomers then became insoluble, resulting in the gel-like precipitate.

### Example 6. Oxidation of Graphimine to Graphamid

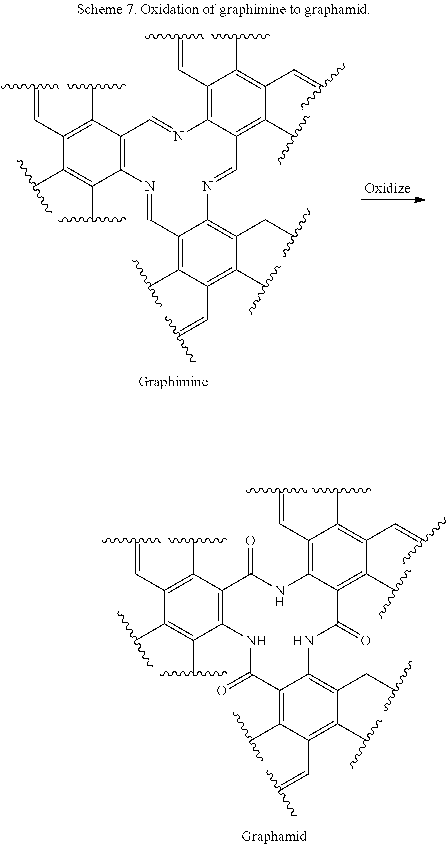 [SMILES: *Cc1c(*)c(C=*)c(*)c2c1/N=C/c1c(*)c(C=*)c(*)c(C=*)c1/N=C/c1c(*)c(C=*)c(*)c(*)c1/N=C/2 ; *Cc1c(*)c(C=*)c(*)c2c1NC(=O)c1c(*)c(C=*)c(*)c(C=*)c1NC(=O)c1c(*)c(C=*)c(*)c(*)c1NC2=O]

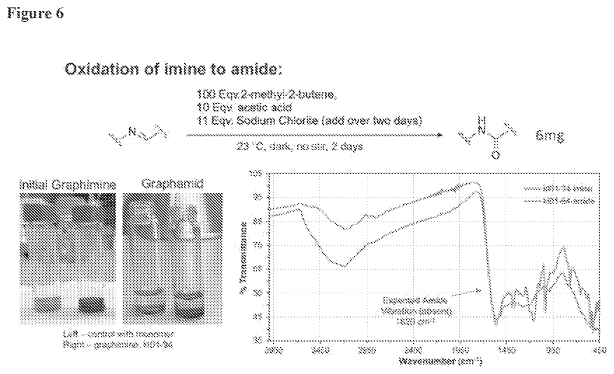

As shown in FIG. 6, graphimine was subjected to oxidation conditions. FTIR analysis shows a reduction in the imine-COFs wavelengths at 1036, 1157, and 1176 cm⁻¹, but lacks the appearance of strong vibrational modes in the amide range at 1620 cm⁻¹.

### REFERENCES

(1) Garcia, J. M.; Garcia, F. C.; Sema, F.; de la Pena, J. L. High-performance aromatic polyamides. Prog. Polymer. Sci. 2010, 35 (5), 623-686. DOI: 10.1016/j.progpolymsci.2009.09.002.
(2) Geng, K.; He, T.; Liu, R.; Dalapata, S.; Tan. K. T.; Li. Z.; Tao, S.; Gong, Y.; Jiang, Q.; Jiang. D. Covalent organic frameworks: design, synthesis, and functions. Chem. Rev. 2020, 120, 8814-8933.
(3) Sandoz-Rosado, E.; Beaudet, T. D.; Andzelm, J. W.; Wetzel, E. D. High strength films from oriented, hydrogen-bonded “graphamid” 2D polymer molecular ensembles. Sci. Rep. 2018, 8, 3708.
(4) Lustig, S. R; Andzelm, J. W.; Wetzel, E. D. Highly thermostable dynamic structure of polyaramid two-dimensional polymers Macromolecules 2021, 54, 1291-1303.
(5) Holst, C.; Schollmeyer, D.; Meier, H. An Efficient Synthesis of Rubin's Aldehyde and its Precursor 1,3,5-Tribromo-2,4,6-tri(dichloromethyl)benzene. Z. Naturforsch. 2011, 935-938.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula (I): 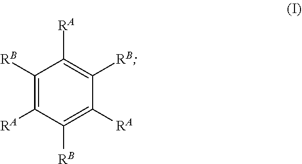
or a salt thereof;
wherein:
R^{A} is selected from the group consisting of —CH═O, —CH═NH, and —CH═NH₂⁺;
R^{B} is selected from the group consisting of —NH₂, —NH₃⁺, and —N═C(R^{C})₂; and
each occurrence of R^{C} is independently selected from the group consisting of optionally substituted aryl and heteroaryl;
all occurrences of R^{A} are the same; and
all occurrences of R^{B} are the same.

2. The compound of claim 1, wherein R^{A} is —CH═O.

3-4. (canceled)

5. The compound of claim 1, wherein R^{B} is —NH₂ or —NH₃⁺.

6. The compound of claim 1, wherein R^{B} is —NH₂.

7. (canceled)

8. The compound of claim 1, selected from the following compounds:

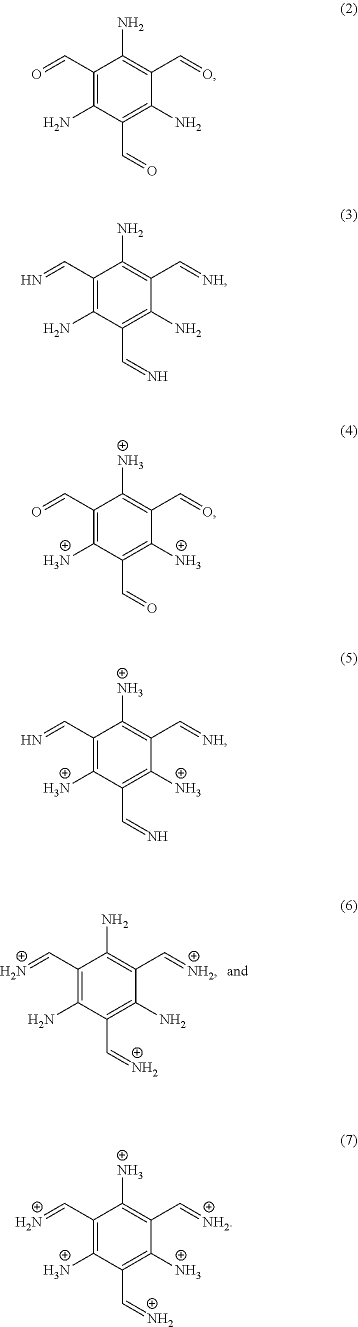

9. (canceled)

10. The compound of claim 1, wherein R^{B} is —N═C(R^{C})₂, optionally wherein each occurrence of R^{c} is phenyl.

11-12. (canceled)

13. The compound of claim 10, wherein the compound is

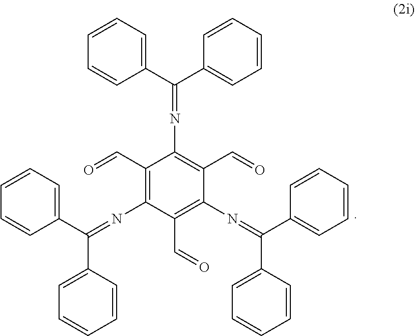 [SMILES: O=Cc1c(N=C(c2ccccc2)c2ccccc2)c(C=O)c(N=C(c2ccccc2)c2ccccc2)c(C=O)c1N=C(c1ccccc1)c1ccccc1]

14. A two-dimensional polymer formed from a plurality of hexavalent monomer units; wherein the hexavalent monomer unit is a compound of claim 5, or

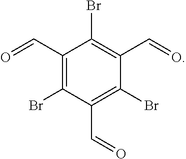

15. (canceled)

16. The two-dimensional polymer of claim 14, wherein the hexavalent monomer units are covalently linked through a linking moiety; optionally wherein each linking moiety has valency of two or greater.

17-18. (canceled)

19. The two-dimensional polymer of claim 16, wherein each linking moiety independently comprises one or more optionally substituted aromatic rings.

20. The two-dimensional polymer of claim 16, wherein each linking moiety is independently selected from the group consisting of

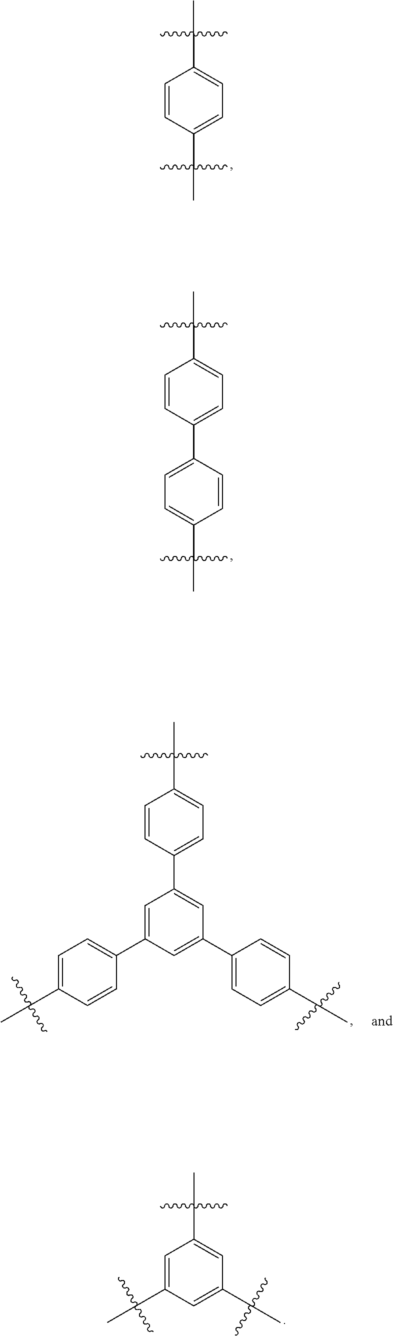 [SMILES: *c1ccc(*)cc1 ; *c1ccc(-c2ccc(*)cc2)cc1 ; *c1ccc(-c2cc(-c3ccc(*)cc3)cc(-c3ccc(*)cc3)c2)cc1 ; *c1cc(*)cc(*)c1]

21-22. (canceled)

23. The two-dimensional polymer of claim 20, having a structure selected from: 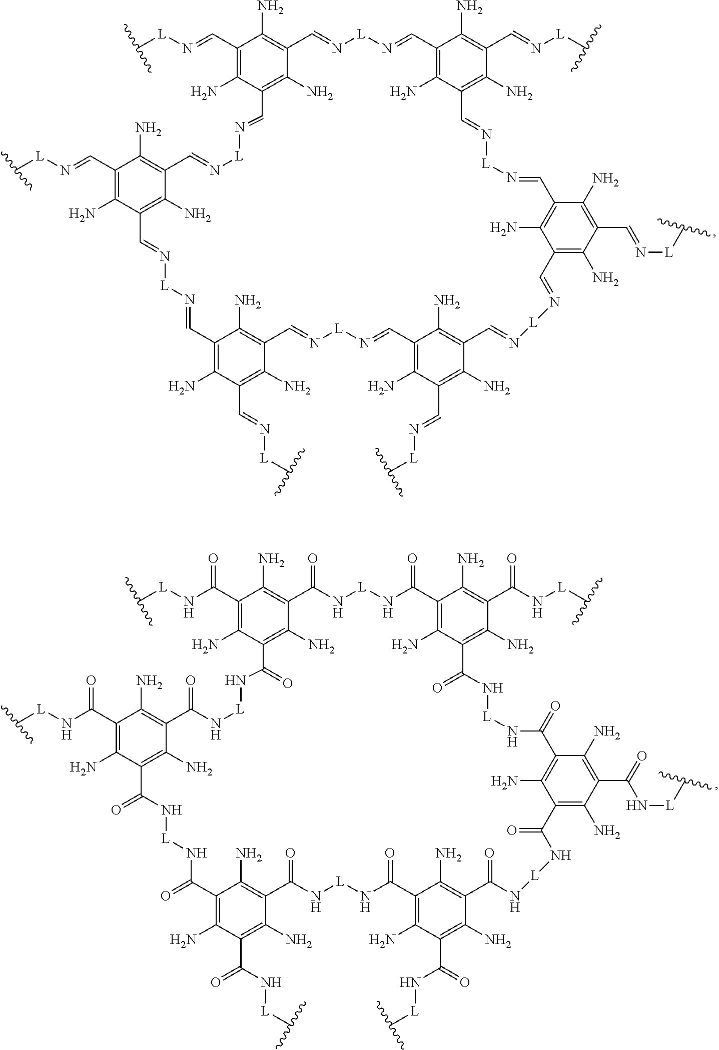 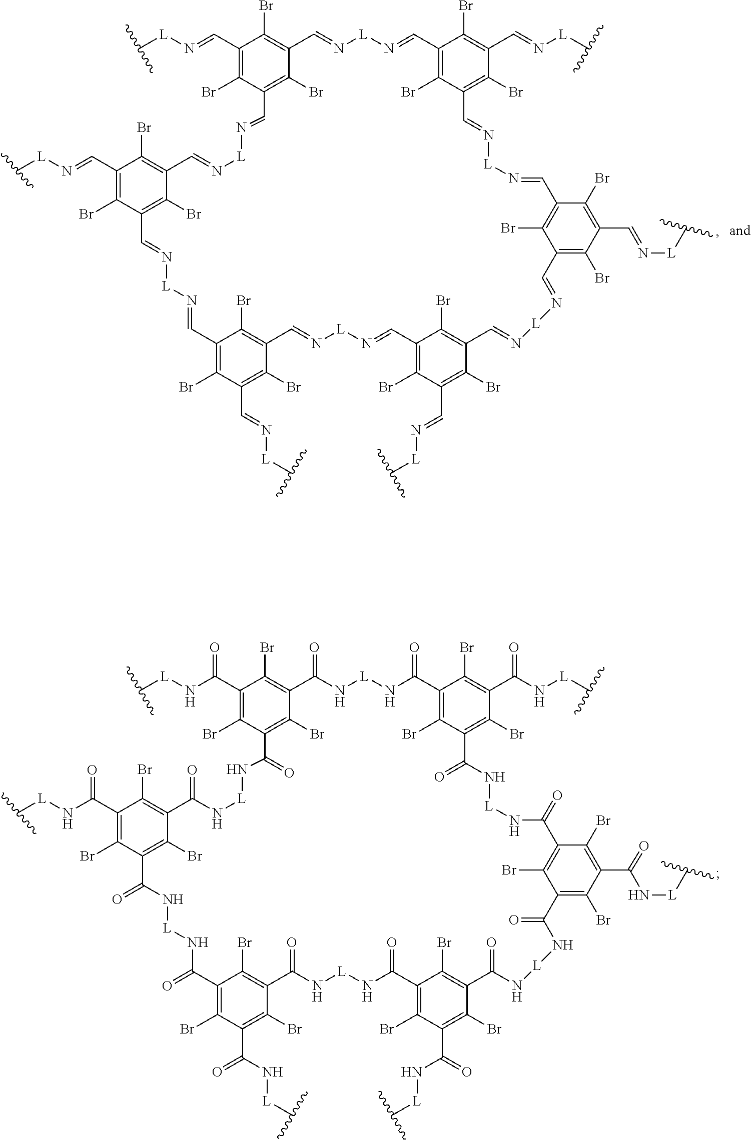
wherein each occurrence of L is

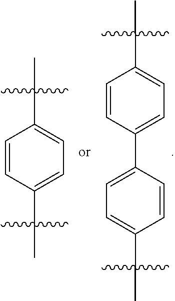

24. (canceled)

25. The two-dimensional polymer of claim 20, having a structure selected from: 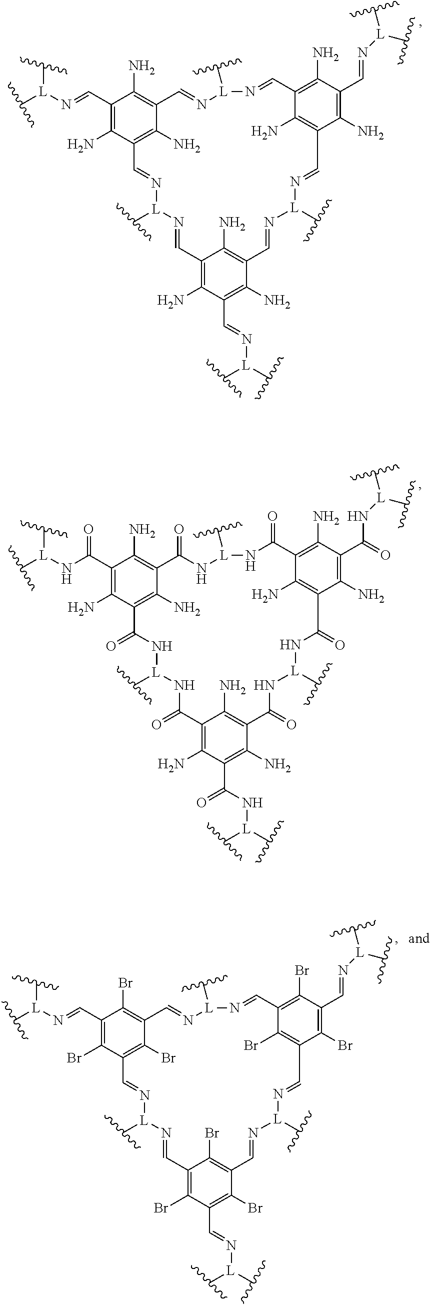 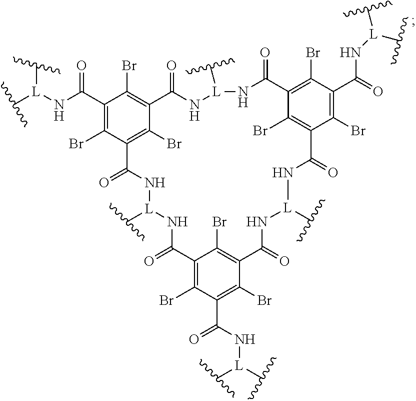
wherein each occurrence of L is

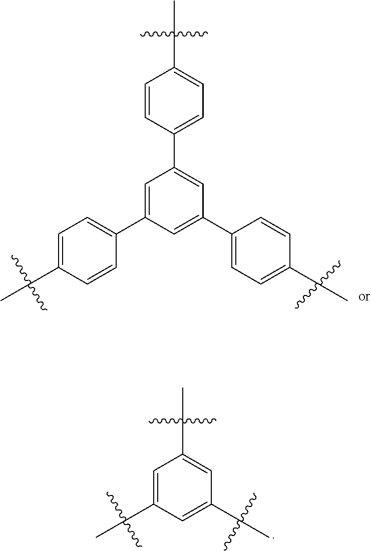 [SMILES: *c1ccc(-c2cc(-c3ccc(*)cc3)cc(-c3ccc(*)cc3)c2)cc1 ; *c1cc(*)cc(*)c1]

26. The two-dimensional polymer of claim 14, wherein the polymer comprises hexagonal pores or triangular pores.

27. (canceled)

28. The two-dimensional polymer of claim 14, wherein the polymer is graphimine:

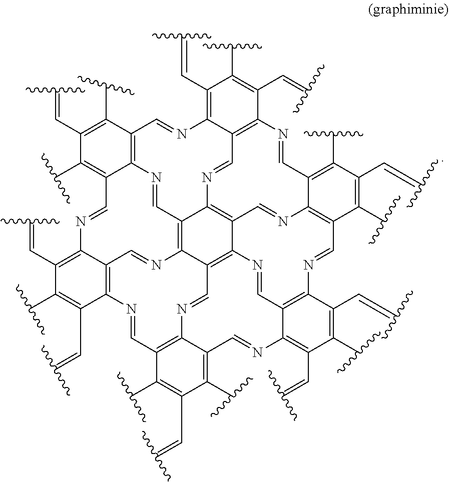 [SMILES: *c1c(C=*)c2ncc3c(*)c(C=*)c(*)c4cnc5c(C=*)c(*)c(C=*)c6ncc7c(*)c(C=*)c(*)c8cnc9c(C=*)c(*)c(C=*)c%10ncc%11c(*)c(C=*)c(*)c%12cnc(c1C=*)c2cnc1c(cnc34)c(ncc56)c(cnc78)c(ncc%109)c1cnc%12%11]

29. A synthetic fiber, comprising a two-dimensional polymer of claim 14.

30. A one-dimensional linear or branched polymer formed from a plurality of hexavalent monomer units; wherein the hexavalent monomer unit is a compound of claim 5, or

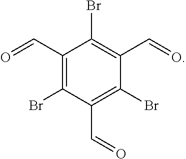

31-33. (canceled)

34. A compound of formula (I-PG): 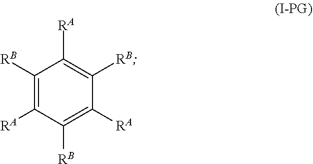
or a salt thereof;
wherein:
R^{A} is selected from the group consisting of —CH═O, —CH═NH, —CH═NH₂⁺, a protected aldehyde, and a protected imine;
R^{B} is selected from the group consisting of —NH₂, —NH₃⁺, —N═C(R^{C})₂, and a protected amine; and
each occurrence of R^{C} is independently selected from the group consisting of optionally substituted aryl and heteroaryl;
wherein:
(a) at least one instance of R^{A} is a protected aldehyde or a protected imine; or
(b) at least one instance of R^{B} is a protected amine.

35-39. (canceled)

40. A method of making the two-dimensional polymer of claim 16, comprising the step of combining under conditions sufficient to produce the two-dimensional polymer:
(i) a compound of formula (I): 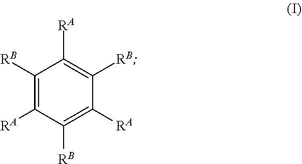
or a salt thereof;
wherein:
R^{A} is selected from the group consisting of —CH═O, —CH═NH, and —CH═NH₂⁺;
R^{B} is selected from the group consisting of —NH₂ and —NH₃⁺;
each occurrence of R^{C} is independently selected from the group consisting of optionally substituted aryl and heteroaryl;
all occurrences of R^{A} are the same; and
all occurrences of R^{B} ae the same; 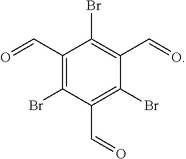 [SMILES: O=Cc1c(Br)c(C=O)c(Br)c(C=O)c1Br]
and
(ii) H₂N-L-NH₂, wherein L represents the linking moiety;
wherein the conditions sufficient to produce the two-dimensional polymer comprise a Bronsted acid.

41. (canceled)

42. A method of making a compound of claim 8, or a salt thereof, comprising the step of combining under conditions sufficient to produce the compound:
compound (1) and a nucleophilic amine source; wherein compound (1) has the following structure: 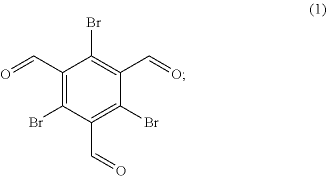 [SMILES: O=Cc1c(Br)c(C=O)c(Br)c(C=O)c1Br]
and
the amine source is selected from the group consisting of ammonia (NH₃), and salts thereof, ammonium salts, primary organic amines, and salts thereof.

43. (canceled)
